SEQUENCE LISTING (93 sequences)

SEQ ID NO: 1 - protein, 9 aa, Homo sapiens
QYNPIRTTF

SEQ ID NO: 2 - protein, 314 aa, Homo sapiens
MYKMEYSYLNSSAYESCMAGMDTSSLASAYADFSSCSQASGFQYNPIRTTFGATSGCPSLTPGSCSLGTLRDHQSSPYAAVPYKLFTDHGGLNEKRKQRRIRTTFTSAQLKELERVFAETHYPDIYTREELALKIDLTEARVQVWFQNRRAKFRKQERAAAAAAAAAKNGSSGKKSDSSRDDESKEAKSTDPDSTGGPGPNPNPTPSCGANGGGGGGPSPAGAPGAAGPGGPGGEPGKGGAAAAAAAAAAAAAAAAAAAAGGLAAAGGPGQGWAPGPGPITSIPDSLGGPFASVLSSLQRPNGAKAALVKSSMF

SEQ ID NO: 3 - protein, 257 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSS

SEQ ID NO: 4 - protein, 108 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTV

SEQ ID NO: 5 - protein, 119 aa, Homo sapiens
EVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSS

SEQ ID NO: 6 - protein, 7 aa, Homo sapiens
DSLGNKY

SEQ ID NO: 7 - protein, 7 aa, Homo sapiens
QDSKRPS

SEQ ID NO: 8 - protein, 12 aa, Homo sapiens
QAWDSSRGYTVV

SEQ ID NO: 9 - protein, 8 aa, Homo sapiens
GFTFDSYA

SEQ ID NO: 10 - protein, 6 aa, Homo sapiens
SGYGGS

SEQ ID NO: 11 - protein, 9 aa, Homo sapiens
YTYFLDAFD

SEQ ID NO: 12 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 21 aa, Homo sapiens
MALPVTALLLPLALLLHAARP

SEQ ID NO: 14 - protein, 119 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQT

SEQ ID NO: 15 - protein, 18 aa, Homo sapiens
GGSGGGGSGGGGSGGGGS

SEQ ID NO: 16 - protein, 121 aa, Homo sapiens
EVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSS

SEQ ID NO: 17 - protein, 45 aa, Homo sapiens
TTTPAPRPPTPAPTIASQPLSLRPEACRPAAGGAVHTRGLDFACD

SEQ ID NO: 18 - protein, 24 aa, Homo sapiens
IYIWAPLAGTCGVLLLSLVITLYC

SEQ ID NO: 19 - protein, 42 aa, Homo sapiens
KRGRKKLLYIFKQPFMRPVQTTQEEDGCSCRFPEEEEGGCEL

SEQ ID NO: 20 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 257 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSQRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSS

SEQ ID NO: 22 - protein, 108 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSQRGYTVVFGTGTKVTV

SEQ ID NO: 23 - protein, 119 aa, Homo sapiens; identical to SEQ ID NO: 5

SEQ ID NO: 24 - protein, 7 aa, Homo sapiens
DSLGNKY

SEQ ID NO: 25 - protein, 7 aa, Homo sapiens
QDSKRPS

SEQ ID NO: 26 - protein, 12 aa, Homo sapiens
QAWDSQRGYTVV

SEQ ID NO: 27 - protein, 8 aa, Homo sapiens
GFTFDSYA

SEQ ID NO: 28 - protein, 6 aa, Homo sapiens
SGYGGS

SEQ ID NO: 29 - protein, 9 aa, Homo sapiens
YTYFLDAFD

SEQ ID NO: 30 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSQRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 119 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSQRGYTVVFGTGTKVTVSSQT

SEQ ID NO: 32 - protein, 121 aa, Homo sapiens; identical to SEQ ID NO: 16

SEQ ID NO: 33 - protein, 257 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGGTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSS

SEQ ID NO: 34 - protein, 108 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSSRGGTVVFGTGTKVTV

SEQ ID NO: 35 - protein, 119 aa, Homo sapiens; identical to SEQ ID NO: 5

SEQ ID NO: 36 - protein, 7 aa, Homo sapiens
DSLGNKY

SEQ ID NO: 37 - protein, 7 aa, Homo sapiens
QDSKRPS

SEQ ID NO: 38 - protein, 12 aa, Homo sapiens
QAWDSSRGGTVV

SEQ ID NO: 39 - protein, 8 aa, Homo sapiens
GFTFDSYA

SEQ ID NO: 40 - protein, 6 aa, Homo sapiens
SGYGGS

SEQ ID NO: 41 - protein, 9 aa, Homo sapiens
YTYFLDAFD

SEQ ID NO: 42 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGGTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 119 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGGTVVFGTGTKVTVSSQT

SEQ ID NO: 44 - protein, 121 aa, Homo sapiens; identical to SEQ ID NO: 16

SEQ ID NO: 45 - protein, 257 aa, Homo sapiens; identical to SEQ ID NO: 21

SEQ ID NO: 46 - protein, 108 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSQRGGTVVFGTGTKVTV

SEQ ID NO: 47 - protein, 119 aa, Homo sapiens; identical to SEQ ID NO: 5

SEQ ID NO: 48 - protein, 7 aa, Homo sapiens
DSLGNKY

SEQ ID NO: 49 - protein, 7 aa, Homo sapiens
QDSKRPS

SEQ ID NO: 50 - protein, 12 aa, Homo sapiens
QAWDSQRGGTVV

SEQ ID NO: 51 - protein, 8 aa, Homo sapiens
GFTFDSYA

SEQ ID NO: 52 - protein, 6 aa, Homo sapiens
SGYGGS

SEQ ID NO: 53 - protein, 9 aa, Homo sapiens
YTYFLDAFD

SEQ ID NO: 54 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSQRGGTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 119 aa, Homo sapiens
SRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSQRGGTVVFGTGTKVTVSSQT

SEQ ID NO: 56 - protein, 121 aa, Homo sapiens; identical to SEQ ID NO: 16

SEQ ID NO: 57 - protein, 249 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYVYFLDAFDIWGQGTMVTVSS

SEQ ID NO: 58 - protein, 108 aa, Homo sapiens; identical to SEQ ID NO: 4

SEQ ID NO: 59 - protein, 119 aa, Homo sapiens
EVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYVYFLDAFDIWGQGTMVTVSS

SEQ ID NO: 60 - protein, 7 aa, Homo sapiens
DSLGNKY

SEQ ID NO: 61 - protein, 7 aa, Homo sapiens
QDSKRPS

SEQ ID NO: 62 - protein, 13 aa, Homo sapiens
CQAWDSSRGYTVV

SEQ ID NO: 63 - protein, 8 aa, Homo sapiens
GFTFDSYA

SEQ ID NO: 64 - protein, 6 aa, Homo sapiens
SGYGGS

SEQ ID NO: 65 - protein, 10 aa, Homo sapiens
YVYFLDAFDI

SEQ ID NO: 66 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYVYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 119 aa, Homo sapiens; identical to SEQ ID NO: 14

SEQ ID NO: 68 - protein, 121 aa, Homo sapiens
EVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYVYFLDAFDIWGQGTMVTVSSSS

SEQ ID NO: 69 - protein, 249 aa, Homo sapiens
QSVLTQPPSVSVSPGQTASITCSGDSLGNAYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSS

SEQ ID NO: 70 - protein, 108 aa, Homo sapiens
QSVLTQPPSVSVSPGQTASITCSGDSLGNAYACWYQQKPGQSPVLVIYQDSKRPSGIPERFSGSNSGNTATLTISGTQAMDEADYYCQAWDSSRGYTVVFGTGTKVTV

SEQ ID NO: 71 - protein, 119 aa, Homo sapiens; identical to SEQ ID NO: 5

SEQ ID NO: 72 - protein, 7 aa, Homo sapiens
DSLGNAY

SEQ ID NO: 73 - protein, 7 aa, Homo sapiens
QDSKRPS

SEQ ID NO: 74 - protein, 12 aa, Homo sapiens
QAWDSSRGYTVV

SEQ ID NO: 75 - protein, 8 aa, Homo sapiens
GFTFDSYA

SEQ ID NO: 76 - protein, 6 aa, Homo sapiens
SGYGGS

SEQ ID NO: 77 - protein, 10 aa, Homo sapiens
YTYFLDAFDI

SEQ ID NO: 78 - protein, 502 aa, Homo sapiens
MALPVTALLLPLALLLHAARPSRNGGDGQSVLTQPPSVSVSPGQTASITCSGDSLGNAYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 119 aa, Homo sapiens
SRNGGDGQSVLTQPPSVSVSPGQTASITCSGDSLGNAYACWYQQKPGQSPVLVIYQDSKRPSGIPERFSGSNSGNTATLTISGTQAMDEADYYCQAWDSSRGYTVVFGTGTKVTVSSQT

SEQ ID NO: 80 - protein, 121 aa, Homo sapiens; identical to SEQ ID NO: 16

SEQ ID NO: 81 - protein, 249 aa, Homo sapiens
QSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGATYYADSVKGRFTISRDNSKNTLYLQMNSLRAEDTAVYYCAKYTYFLDAFDIWGQGTMVTVSS

SEQ ID NO: 82 - protein, 249 aa, Homo sapiens; identical to SEQ ID NO: 81

SEQ ID NO: 83 - protein, 119 aa, Homo sapiens
EVQLLESGGGLVQPGGSLRLSCAASGFTFDSYAMSWVRQAPGKGLEWVSAISGYGGATYYADSVKGRFTISRDNSKNTLYLQMNSLRAEDTAVYYCAKYTYFLDAFDIWGQGTMVTVSS

SEQ ID NO: 84 - protein, 7 aa, Homo sapiens
DSLGNKY

SEQ ID NO: 85 - protein, 7 aa, Homo sapiens
QDSKRPS

SEQ ID NO: 86 - protein, 12 aa, Homo sapiens
QAWDSSRGYTVV

SEQ ID NO: 87 - protein, 8 aa, Homo sapiens
GFTFDSYA

SEQ ID NO: 88 - protein, 6 aa, Homo sapiens
SGYGGA

SEQ ID NO: 89 - protein, 10 aa, Homo sapiens
YTYFLDAFDI

SEQ ID NO: 90 - protein, 502 aa, Homo sapiens
MALPVTALLLPLALLLHAARPSRNGGDGQSVLTQPPSVSVSPGQTASITCSGDSLGNKYACWYQQKPGQSPVLVIYQDSKRPSGIPERFSGSNSGNTATLTISGTQAMDEADYYCQAWDSSRGYTVVFGTGTKVTVSSQTGGSGGGGSGGGGSGGGGSEVQLLESGGGLVQPGGSLRLSCAASGFTFDSYAMSWVRQAPGKGLEWVSAISGYGGATYYADSVKGRFTISRDNSKNTLYLQMNSLRAEDTAVYYCAKYTYFLDAFDIWGQGTMVTVSSSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 119 aa, Homo sapiens; identical to SEQ ID NO: 14

SEQ ID NO: 92 - protein, 121 aa, Homo sapiens
EVQLLESGGGLVQPGGSLRLSCAASGFTFDSYAMSWVRQAPGKGLEWVSAISGYGGATYYADSVKGRFTISRDNSKNTLYLQMNSLRAEDTAVYYCAKYTYFLDAFDIWGQGTMVTVSSSS

SEQ ID NO: 93 - protein, 119 aa, Homo sapiens
EVQLLESGGGLVQPGGSLRLSCAASGFTFDSYAMSWVRQAPGKGLEWVSAISGYGGSTYYADSVKGRFTISRDNSKNTLYLQMNSLRAEDTAVYYCAKYTWFLDAFDIWGQGTMVTVSS

(19) United States
(12) Patent Application Publication
(10) Pub. No.: US 2026/0243766 A1
(43) Pub. Date: Aug. 20, 2026

(54) IMPROVED PHOX2B PC-CAR GENERATION BASED ON STRUCTURE AND SATURATION MUTAGENESIS

(71) Applicant: THE CHILDREN'S HOSPITAL OF PHILADELPHIA, Philadelphia, PA (US)

(72) Inventor: Nikolaos SGOURAKIS, Philadelphia, PA (US)

(73) Assignee: THE CHILDREN'S HOSPITAL OF PHILADELPHIA, Philadelphia, PA (US)

(21) Appl. No.: 19/159,543

(22) PCT Filed: Feb. 23, 2024
(86) PCT No.: PCT/US2024/017081

§ 371 Date: Aug. 25, 2025

Related U.S. Application Data
(60) Provisional application No. 63/486,910, filed on Feb. 24, 2023.

(51) Int. Cl. G01N 33/569; G01N 33/543

(52) U.S. Cl. CPC G01N 33/56977; G01N 33/54313; G01N 2333/70539

(57) ABSTRACT

The neuroblastoma immunopeptidome is enriched with peptides derived from proteins essential for tumorigenesis including the unmutated peptide QYNPIRTTF (SEQ ID NO: 1) discovered on HLA-A*24:02 which is derived from the neuroblastoma dependency gene and master transcriptional regulator PHOX2B. To target QYNPIRTTF, a saturation mutagenesis screen of the scFV CDR loops of a previously developed peptide-centric chimeric antigen receptors (PC-CARs) was performed to identify improved immunotherapeutics targeting of the neuroblastoma CRC master regulator PHOX2B. Also provided are methods of screening for cross-reactivity of a candidate peptide:HLA targeted therapeutic against off-target HLAs.

### PRIORITY CLAIM

This application claims benefit of priority to U.S. Provisional Application Ser. No. 63/486,910, filed Feb. 24, 2023, the entire contents of which are hereby incorporated by reference.

### STATEMENT REGARDING FEDERALLY FUNDED RESEARCH

This invention was made with government support under grants AI143997, R35 GM125034, U54 CA232568, R35 CA220500 and DK112217 awarded by National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Feb. 22, 2024, is named CHOP.P0065WO—Sequence Listing.xml and is ~88 kilobytes in size.

### FIELD

The present disclosure relates generally to binding agents and methods of use therefor for diagnosing and/or treating cancer. In particular, the disclosure relates to binding agents capable of specifically binding to peptide:MHC complexes displaying peptides derived from human PHOX2B as well as method of screening for cross-reactivity of a candidate peptide:HLA targeted therapeutic against off-target HLAs.

### BACKGROUND

The major histocompatibility (MHC) system, also known as known as human leukocyte antigens (HLA) in humans, presents peptide antigens to T cells. Recognition by T cells of specific peptide:MHC (pMHC) complexes is mediated by the T-cell receptor (TCR). This recognition contributes to antigen-specific expansion of T cells and other immunological effects. From a biomedical perspective, pMHC complexes represent potential markers for disease status and targets for immunotherapy. Binding agents that recognize pMHC complexes have proven difficult to generate by conventional methods. One reason for this difficulty is the nature of the epitope. A pMHC-specific binding agent must recognize structural features of the peptide antigen as well as the MHC molecule. Typical pMHC-displayed peptide antigens are only 9 to 12 residues in length with some of those residues buried within the pMHC complex. This limits the number of surface-accessible residues that are free to interact with a binding agent. Typical antibody epitopes involve a greater number of amino acid residues than the peptide antigen alone provides. For a binding agent to achieve specific binding to a pMHC complex, either the epitope must be smaller than typically required for specific binding or the epitope must encompass portions of the MHC molecule in addition to the peptide. Put another way, antibodies that bind only to the peptide without expanding the epitope to the MHC molecule will in most cases lack the affinity required for a useful pMHC binding agent. Thus, the epitope for pMHC binding agents will in most cases extend to the MHC molecule.

However, if the interaction between the binding agent and pMHC complex depends too much upon interactions with the MHC molecule, then the binding agent will bind non-specifically to pMHC complexes displaying non-target peptide antigens. Finally, binding agents that bind the MHC independent of the peptide displayed are incapable of distinguishing MHC complexes displaying other peptides from pMHC complexes of interest.

From a theoretical perspective, native TCRs should have overcome this specificity problem and should prove useful as binding agents for pMHC complexes. This has not, however, proven to be the case. Native TCR receptors have been cloned from epitope-reactive T cell populations. Studies of these cloned TCRs have demonstrated that they have surprisingly low affinity for their cognate pMHC complexes—typically micromolar (μM) dissociation constants. Also, native TCRs rarely have the binding specificity necessary for practical use as binding agents for pMHC complexes, with a single TCR able to recognize many different epitopes and selectivity within the immune system achieved by deleting self-reactive T-cells or making them anergic.

In contrast to TCRs, antibodies generally have much higher affinities for their targets but efforts to raise antibodies against pMHC complexes have similarly foundered. Traditional techniques for antibody discovery, namely, animal vaccination or library screening, rarely succeed in generating pMHC binding agents with useful binding characteristics.

Neuroblastoma is a childhood cancer derived from tissue of the developing sympathetic nervous system and is often lethal despite intensive cytotoxic therapy (Matthay et al., 2016). These tumors are low in mutational burden (Pugh et al., 2013; Schramm et al., 2015; Cheung et al., 2012; Molenaar et al., 2012; Brady et al., 2020) and MHC expression (Woelfl et al., 2005; Burr et al., 2019), making neuroblastoma both a challenging tumor to target with MHC-based immunotherapies. Previous work had identified PHOX2B as being highly and specifically expressed in neuroblastoma tissues (Andreatta et al., 2012), as well as being mutated in cases of familial inheritance of the disease (Alvarez et al., 2019; Sci Adv, 2021).

Consistent with its function in orchestrating neural crest progenitor development (Pattyn et al., 1999; Dauger et al., 2003), PHOX2B is expressed exclusively during fetal development and is completely silenced in normal tissues prior to birth. PHOX2B expression is routinely used in neuroblastoma diagnostic assays (Hata et al., 2015; Hung et al., 2017), is one of two highly penetrant susceptibility genes in neuroblastoma (Mosse et al., 2004), and is the third most significant dependency in neuroblastoma as reported in DepMap (Tsherniak et al., 2017; Dharia et al., 2021). Taken together, the inventors suggest that PHOX2B is a highly specific tumor antigen in neuroblastoma and an ideal candidate for therapeutic targeting.

The patent WO2019/178081 analyzed the immunopeptidome (the repertoire of peptides displayed as peptide-MHC complexes) of 16 neuroblastoma tumors and determined a number of peptides that were presented by different human peptide-MHC alleles, among which was a PHOX2B-derived peptide, QYNPIRTTF (SEQ ID NO: 1), presented by the HLA-A*24:02 subtype.

A binding agent specific to this HLA-A*24:02 PHOX2B peptide complex would meet a great unmet need to treat and diagnose neuroblastoma in pediatric patients.

### SUMMARY

Provided herein are methods screening for cross-reactivity of a candidate peptide:HLA targeted therapeutic against off-target HLAs, the method comprising the steps of: (a) contacting an antigen-binding portion of the candidate peptide:HLA targeted therapeutic with a composition comprising human leukocyte antigens linked to a solid substrate, and (b) detecting binding of the antigen-binding portion of the candidate peptide:HLA targeted therapeutic to the solid substrate, wherein binding of the antigen-binding portion of the candidate peptide:HLA targeted therapeutic to the solid substrate is indicative of a cross-reactivity specific for said native human leukocyte antigens.

The human leukocyte antigens linked to the solid substrate may be peptide-loaded. The human leukocyte antigens linked to the solid substrate may have been incubated with an excess of a target peptide (i.e., the peptide towards which the peptide:HLA targeted therapeutic is directed) prior art step (a). The target peptide may be peptide to which the candidate peptide:HLA targeted therapeutic is targeted. The composition comprising the human leukocyte antigens linked to a solid substrate may comprise at least 90% native human leukocyte antigens and at most 10% denatured human leukocyte antigens. The antigen-binding portion of the candidate peptide:HLA targeted therapeutic may be an scFV. The human leukocyte antigens may be selected from the group consisting of class I human leukocyte antigens, class II human leukocyte antigens and combinations thereof. The human leukocyte antigens may be class I human leukocyte antigens. The solid substrate may be selected from the group consisting of a plurality of beads, a plurality of microbeads, a plurality of microparticles, a plurality of microspheres, a well, a membrane, a polymer, a filter and a microarray and combinations thereof. The solid substrate may be a plurality of microbeads. The solid substrate may comprise a material selected from the group consisting of silica, gold, latex, polystyrene, polysulfone, hydrogel, polyvinyl chloride, glass, and combinations thereof. The solid substrate may comprise a detectable label. The detectable label is a fluorescent dye, a radioactive label, a magnetic label, a bar code, or combinations thereof. The human leukocyte antigens may be covalently linked to the solid substrate. The composition may comprise a plurality of said solid substrates, wherein at least 90% of the human leukocyte antigens linked to a particular solid substrate of the plurality are of the same allele and each solid substrate of the plurality is linked to a different human leukocyte antigen allele with respect to the other solid substrates of the plurality. The native and denatured human leukocyte antigens may be class I human leukocyte antigens. The plurality of solid substrates may comprise four or more solid substrates, eight or more solid substrates, 16 or more solid substrates, or 32 or more solid substrates. The detecting binding of the antigen-binding portion of the candidate peptide:HLA targeted therapeutic may be performed using flow cytometry. The detecting binding of the antigen-binding portion of the candidate peptide:HLA targeted therapeutic may be performed using a secondary antibody. The secondary antibody may comprise a label selected from the group consisting of a radioactive label, a fluorescent label, an enzymatic label, an avidin label, a biotin label and combinations thereof. The solid substrate may comprise a plurality of microbeads or microparticles. The solid substrate may comprise a plurality of microbeads, wherein each microbead of the plurality comprises a detectable label. The detectable label is a fluorescent dye, a radioactive label, a magnetic label, or a bar code.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Schematic representation of the SABs assay used to measure the binding levels of scFv binders. The SABs are color coded and coated with a variety of HLA-I allotypes. Binding levels were evaluated by incubation with soluble scFv monomer tagged with 6×-His followed by detection using a PE-conjugated anti-His antibody.

FIGS. 2A-D. Binding levels of the engineered scFV 10LH using SABs. (FIG. 2A) Levels of folded MHCI molecules captured on the SABs using the PE-conjugated W6/32 antibody (Biolegend, 311406). Similar levels of peptide-loaded MHC-I molecules were observed across all the different HLA allotypes. Bar graphs showing the logarithm of Mean Fluorescence Intensity (MFI) levels upon incubation with (FIG. 2B) the PE-conjugated anti-His tag antibody (Biolegend, 362603) and (FIG. 2C) 10LH incubation with or without addition of excess of PHOX2B peptide. (FIG. 2D) Bar graph showing Mean Fluorescent Intensities after subtraction of background staining levels shown in (FIG. 2C). Data shown are averages from n=2 experiments, with errors shown.

FIG. 3. Workflow of site directed mutagenesis screening. PCR amplification is used to mutate key residues of the CDR loops of the CAR scFv to all amino acids. After FACS sorting of single antigen-specific clones, clones were sequenced for mutation identification and functional validation of cytotoxicity and specificity.

FIG. 4. A screen of CDR3 loops identifies clones that resulted in shift toward single specificity. After mutagenesis of the CDR loops, single clones of Jurkat T cells expressing the 10LH.BBz chimeric antigen receptor (CAR) were stained with PHOX2B/HLA-A*24:02 and cross-reactive counterstain CNGB3/HLA-A*24:02 dextramers and analyzed via flow cytometry. Clones demonstrating PHOX2B antigen specific were sorted using FACS.

FIG. 5. Two clones in Light chain CDR3 position 9 and 10 resulted in single-specificity binding to PHOX2B when compared to homologous CNGB3 peptide. Following the procedures outlined previously, mutagenesis clones in light chain CDR3 position 9 and 10 demonstrated specificity to PHOX2B when counterstained with the WT cross-reactive CNGB3 peptide.

FIG. 6. Quantifying the abrogation of cross-reactivity. Antigen specific population frequencies were compared between mutagenesis clones and 10LH.BBz WT CAR T cells using flow cytometry analysis. Fold shifts were calculated from PHOX2B antigen specific population frequencies divided by cross reactive, counterstain population frequencies.

FIG. 7. Characterizing the shift in cross-reactivity of variants across sCRAP/X-scan peptides. Following the procedures outlined previously, mutagenesis clones were stained with PHOX2B and counterstained with known cross-reactive peptides determined from X-scan/ScanProsite and sCRAP.

FIG. 8. Mutagenesis amino acid identification was determined using PCR amplification methods. mRNA was recovered from single cells and populations grown from single cells, and reverse transcription was performed to synthesize cDNA. CDR loops of interest were then amplified using PCR. In addition, gDNA was recovered from single cells and populations grown from single cells, and nested PCR amplification was performed to amplify CDR loops of interest.

FIG. 9. Building a P4 selectivity filter by point mutations of key 10LH CDR3H residues. Modeling using the crystal structure of the 10LH scFv in complex with PHOX2B/HLA-A*24:02 suggests that the Y229W substitution on 10LH can lead to improve peptide specificity through steric hindrance. The spaces of possible cross-reactive peptide sequences are shown as regular expressions for the WT and Y229W variants. Upon Y229W mutation, three residues are allowed at position four of the PHOX2B 9mer QYNPIRTTF: A, G, and P, as opposed to 20 for WT 10LH.

FIG. 10. Perturbing interactions with HLA “framework” residues to abrogate 10LH off-target cross-reactivity. This strategy is leveraging the finding that cross-reactive interactions with peptides (e.g., ATG2A) or HLAs (e.g., HLA-C*07:02) are suboptimal (micromolar range) binders (versus nanomolar range interaction with HLA-A*24:02 PHOX2B). The inventors aim to introduce point mutations on 10LH which weaken the HLA:10LH interface based on the 3D complex structure, such that any further perturbations to the interaction surface would abrogate off-target binding. This would likely also weaken interactions with the target HLA-A*24:02/PHOX2B target, from nanomolar to micromolar range K_{D} (dissociation constant), however, the data suggest that up to approx. 20 micromolar K_{D} interactions with pHLAs can elicit potent CAR-T killing, dependent upon the antigen density (e.g., as seen for A*23:01/PHOX2B). Three examples of framework amino acid substitutions for 10LH are shown on the 3D structure of the HLA-A*24:02/PHOX2B/10LH complex.

FIG. 11. Molecular mimcry of the original PHOX2B/HLA-A*24:02 surface can lead to off-target cross-reactivity of 10LH. Schematic description of how peptides which lack an Arg at position 6 can be recognized by 10LH through compensatory Arg polymorphisms at HLA framework interaction sites. K_{D} (dissociation constant) values measured by SPR for HLA-A*24:02 complexes refolded with Ala substitutions of the PHOX2B peptide (in cyan), showing that the R6A mutations leads to complete loss of binding. In these experiments, a Streptavidin chip surface conjugated with biotinylated 10LH was offered graded concentrations of different HLA protein samples and data were fit using kinetic of equilibrium dissociation models to extract K_{D} values. Structural modeling of the 10LH/PHOX2B^{R6A}/A*24:02^{2A69R} or 10LHPHOX2B^{R6A}/A*24:02^{A69R} demonstrates plausible molecular interactions with the Arg sidechains, which can rescue binding in vitro. SPR-measured K_{D} values for the respective protein complexes are indicated.

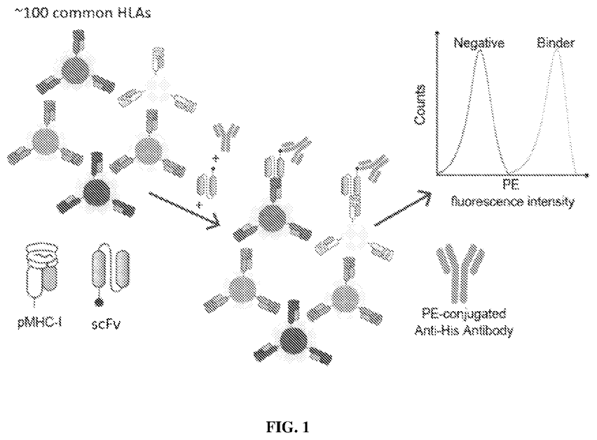

### DETAILED DESCRIPTION

The curative potential of chimeric antigen receptor (CAR) T cell-based cancer immunotherapies has been established in leukemias, but solid tumor applications have been limited by a paucity of known tumor-specific membrane proteins (Mirzaei et al., 2017; Majzner et al., 2019). Though membrane proteins represent up to a quarter of the proteome, only a fraction of these are specifically expressed on tumors cells and not on normal tissues, and a smaller proportion are essential to tumor homeostasis (Tsherniak el al., 2017). Rather, the vast majority of cancer driver proteins reside in the cytoplasm or nucleus of the cell, where they are accessible to the immune system only through presentation of peptides on the major histocompatibility complex (MHC).

MHC class I proteins, encoded by the highly polymorphic human leukocyte antigen (HLA) A, B, and C genes, present a snapshot of the intracellular proteome on the cell surface (immunopeptidome) where T cells surveil the peptide-MHC complexes (pMHC) for antigens derived from foreign pathogens (Germain & Margulies, 1993). T cell recognition of mutation-derived pMHCs (neoantigens) as non-self is the basis of curative responses achieved through immune checkpoint blockade (Crittenden et al., 2018) and complete remissions using adoptive transfer of tumor infiltrating lymphocytes (TILs) (Yossef et al., 2018). Nonetheless, only ~5% of these neoantigens are predicted to bind a given HLA allotype (Yarmarkovich et al., 2020), and just 1.6% of neoantigens are reported to be immunogenic (Leko & Rosenberg, 2020). Subclonal mutations and downregulation of mutated non-essential genes further constrain the pool of therapeutically relevant neoantigens, necessitating a mutational threshold for effective neoantigen-based therapies that is not surpassed in most cancers (Rosenthal et al., 2019; Schumacher & Schreiber, 2015). Tumor cells also present a plethora of unmutated self-peptides on MHC (Shao et al., 2018), but these are largely immunogenically silent due to negative thymic selection of T cells. The inventors hypothesized that a subset of the immunopeptidome consists of tumor-specific peptides derived from essential oncoproteins and that these can be targeted using synthetic peptide-centric chimeric antigen receptors (PC-CARs).

Peptides presented in the MHC groove make up only a small fraction of the extracellular pMHC molecular surface. The typical 8-14mer peptide presented on MHC class I composes only ~2-3% of the amino acids in the pMHC complex and is spatially confined within the adjacent alpha-helices of the MHC groove, thus posing major challenges for engineering peptide-specific single-chain antibody variable fragment (scFv) binders (Maus et al., 2016). Furthermore, cross-reactivity of engineered receptors with peptides of biophysically similar molecular surfaces presented in normal tissues have resulted in significant toxicity and death (Parkhurst et al., 2011; Morgan et al., 2013; Linette et al., 2013).

Here, the inventors present improved immunotherapeutic targeting of the neuroblastoma CRC master regulator PHOX2B using PC-CARs specific against the PHOX2B peptide QYNPIRTTF (SEQ ID NO: 1). Also provided are methods of screening for cross-reactivity of a candidate peptide:HLA targeted therapeutic against off-target HLAs. These and other aspects of the disclosure are set out in detail below.

### I. DEFINITIONS

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, antibodies and related molecules, cell and tissue culture, molecular biology, and protein and oligo- or polynucleotide chemistry and hybridization described herein are those well-known and commonly used in the art.

Standard techniques are used for recombinant DNA, oligonucleotide synthesis, and cell culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See, e.g., B. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); and F. M. Ausubel et al. (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory (1988); and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md. (1987 and 1991); Bork et al., J Mol. Biol. 242, 309-320 (1994); Chothia and Lesk J. Mol Biol. 196:901-917 (1987), Chothia et al. Nature 342, 877-883 (1989), and/or or Al-Lazikani et al. J Mol Biol 273, 927-948 (1997).

As used herein, “PHOX2B” is defined as that sequence disclosed in Uniprot ID Q99453, shown below:

(SEQ ID NO: 2)
MYKMEYSYLNSSAYESCMAGMDTSSLASAYADFSSCSQASGFQYNPIRTTFGATSGCPSLTP
GSCSLGTLRDHQSSPYAAVPYKLFTDHGGLNEKRKQRRIRTTFTSAQLKELERVFAETHYPD
IYTREELALKIDLTEARVQVWFQNRRAKFRKQERAAAAAAAAAKNGSSGKKSDSSRDDESKE
AKSTDPDSTGGPGPNPNPTPSCGANGGGGGGPSPAGAPGAAGPGGPGGEPGKGGAAAAAAAA
AAAAAAAAAAAAGGLAAAGGPGQGWAPGPGPITSIPDSLGGPFASVLSSLQRPNGAKAALVK
SSMF

The PHOX2B epitope that is targeted by the sequences claimed by the invention comprises residues 43-51 of Uniprot ID Q99453 and may be referred to hereafter as PHOX2B 43-51.

As used herein, “antigen-binding site” shall be taken to mean a structure formed by a protein that is capable of binding or specifically binding to an antigen, such as an antibody. The antigen-binding site need not be a series of contiguous amino acids, or even amino acids in a single polypeptide chain. For example, in a Fv comprising two different polypeptide chains from an antibody, the antigen-binding site is made up of a series of amino acids of a V_{L} and a V_{H} that interact with the antigen and that are generally, however not always in one or more of the CDRs in each variable region. In some embodiments, the antigen-binding site is an antigen-binding site of an antibody. In such embodiments, the antigen-binding site may comprise one or more complementarity-determining regions or “CDRs”. In some embodiments, the antigen-binding site of an antibody comprises at least part of a V_{H} or a V_{L} or a Fv.

As used herein, the terms “complementarity-determining region” or “CDR” are used interchangeably to refer to the antigen binding regions found within the variable region of the heavy and light chain polypeptides. Generally, antibodies comprise three CDRs in each of the V_{H} (CDR H1 or H1; CDR H2 or H2; and CDR H3 or H3) and three in each of the V_{L} (CDR L1 or L1; CDR L2 or L2; and CDR L3 or L3).

As used herein, the “variable regions” and “CDRs” may refer to variable regions and CDRs defined by any approach known in the art, including combinations of approaches. According to a specific embodiment, the CDRs are determined according to Kabat et al. (supra).

As used herein “binding” or “binds” or “specifically binds” refers to an antibody:antigen mode of binding, which preferably, in the case of clinically relevant binding agents, means a K_{D} below 1 μM or below 500 nM. The binding agents of the disclosure can bind PHOX2B:pMHC complexes with a high affinity. For example, in some embodiments the binding agent can bind PHOX2B:pMHC with a dissociation constant (K_{D}) equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴. Specificity of binding is determined with reference to non-target proteins, such as for example bovine serum albumin (BSA). In some embodiments, the binding agent binds PHOX2B:pMHC complexes with a dissociation constant (K_{D}) at least 2, 3, 4, 5, 10, 20, 50, 100, 200, 500, 1000, 2000, 5000, 10⁴, 10⁵ or 10⁶-fold lower than the binding agent's dissociation constant for BSA, when measured at physiological conditions. In some cases, specificity is determined by measuring binding of a binding agent to an MHC that is loaded with a non-target peptide or that is empty.

In some cases, specificity is determined by measuring binding of a binding agent to the target peptide alone or the target peptide loaded on an MHC of a different allotype. In particular embodiments of the present disclosure, the binding agent is MHC-restricted which means that the binding agent binds specifically to a target peptide (e.g., PHOX2B peptide) loaded onto an MHC representative of a chosen allelic variant (e.g., HLA-A*24:02) with a dissociation constant (K_{D}) at least 2, 3, 4, 5, 10, 20, 50, 100, 200, 500, 1000, 2000, 5000, 10⁴, 10⁵ or 10⁶-fold lower than the binding agent's dissociation constant for an MHC from another allelic variant.

As used herein the phrase “chimeric antigen receptor (CAR)” refers to a recombinant or synthetic molecule which combines antibody-based specificity for a desired antigen with a T cell receptor-activating intracellular domain to generate a chimeric protein that exhibits cellular immune activity to the specific antigen.

As used herein the phrase “T Cell Receptor” or “TCR” refers to soluble and non-soluble forms of recombinant T-cell receptor.

As used herein, a “T-cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell.

As used herein a “T Cell Receptor-like antibody” or “TCRL” refers to an antibody which binds an MHC displaying an HLA-restricted peptide antigen. Binding of the TCRL to its target typically has an MHC-restricted specificity: the TCRL does not bind the MHC in the absence of the complexed peptide, and the TCRL does not bind the peptide in an absence of the MHC. TCRLs are characterized by affinity sufficient to permit specific binding to a tumor antigen even when the TCRL is provided in a soluble, rather than membrane-bound, form. TCRLs are being developed as a new therapeutic class for targeting tumor cells and mediating their specific killing. In addition, TCRLs are valuable research reagents enabling the study of human class I peptide-MHC ligand presentation and TCR-peptide-MHC interactions. In an embodiment, the binding agent of the present disclosure is a TCRL.

As used herein the phrase “MHC (or HLA)-restricted peptide” refers to a peptide which is potentially presented on an MHC molecule. Such peptides may be identified by laboratory procedures such as Mass-Spectrometry, reverse-immunology or by in-silico analysis. An MHC (or HLA)-presented peptide refers to a peptide which is confirmed in vitro or in vivo as being presented by an MHC molecule.

The term “cancer” as used herein is defined as disease characterized by the rapid and uncontrolled growth of aberrant cells. Cancer cells can spread locally or through the bloodstream and lymphatic system to other parts of the body.

A “compound” refers to any molecule including small molecules, polypeptides, and other macromolecules. In some embodiments, a compound is a small molecular weight compound with a molecular weight of less than about 2000 Daltons.

The term “naturally occurring” (or “native”) as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally occurring.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical linker or a disulphide bond, for example. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics.

The term “agent” is used herein to denote a chemical compound, a mixture of chemical compounds, a biological macromolecule, or an extract made from biological materials.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

### II. BINDING AGENTS

The term “binding agent”, as used herein, refers to any molecule which is capable of binding to the PHOX2B:HLA complex. In particular, the binding agent is capable of binding to a PHOX2B:HLA complex comprising the sequence QYNPIRTTF (SEQ ID NO: 1). In some embodiments, the binding agent is or comprises a polypeptide. In some embodiments, the binding agents of the present disclosure comprise the sequences provided and variants thereof.

The disclosure specifically contemplates binding agents that have at least 75, 76, 77, 78, 79, 80, 81, 82, 83, 84, 85, 86, 87, 88, 89, 90, 91, 92, 93, 94, or 95% sequence identity or even at least 96, 97, 98, or 99% sequence identity to full length variable regions for constructs disclosed here, as long as the binding affinity to the PHOX2B:HLA complex is maintained. In some embodiments, the binding agents of the present disclosure further comprise dimeric binding agents derived by splitting the single chain variable fragment (scFv) sequences into light chain and heavy chain, respectively, at the poly-G/S linker, as well as homologs or variants of such dimeric binding agents. Optionally, specificity or affinity of binding to the PHOX2B:HLA complex is maintained or even improved. In particular embodiments, the binding agent comprises a heavy chain and a light chain which comprises three heavy chain CDR and three light chain CDR sequences, respectively, of the present disclosure, maintaining or improving binding. In embodiments of the disclosure, the binding agent is an antibody, or antigen-binding fragment thereof, an artificial protein that is soluble (e.g., a bispecific antibody), or an artificial protein that is membrane-tethered (e.g., a chimeric antibody receptor or a TCR fusion protein).

For binding agents derived from immunoglobulin (Ig) variable domains, with the target contact surface created through the loops connecting β-strands (the complementarity-determining regions, or CDRs), the binding activity to the target may be transferable through the grafting of the CDR loops to related Ig domains (e.g., other human Ig family members) or even non-Ig β-sheet scaffolds. This is especially the case where the structure of the binding agent in complex with the target indicates that the binding is mainly contributed through one, or a few, of the 6 CDRs of the combined V_{L} and V_{H} domains. The disclosure specifically contemplates binding agents that have at least 50, 55, 60, 65, 70, 75, 80, 85, 90 or 95% sequence identity to single CDR regions for constructs disclosed here, as long as the binding affinity to the PHOX2B:HLA complex is functionally maintained. CDR grafting has been used extensively to ‘humanize’ antibodies where the CDR loops from antibodies derived from a non-human host are grafted onto a human Ig scaffold to reduce immunogenicity. Many antibodies approved for therapeutic use have been humanized through CDR transplantation from murine antibodies onto human scaffolds. Examples where the grafting of CDR loops from antibody scaffolds onto non-Ig alternative scaffolds have also been reported (Nicaise M., et al. Protein Sci 13:1882-1891 (2004); Petrovskaya L E, et al. Biochemistry (Mosc) 77:62-70 (2012); Pacheco et al. Protein Eng Des Sel 27:431-438 (2014)).

Various means of determining the K_{D} of a binding agent for its target are known, including enzyme-linked immunoabsorbance (ELISA) assays and Surface Plasmon Resonance (SPR) assays. In some cases, binding affinity and specificity is determined by optical interferometry, such as with the Pall ForteBio BLItz® system, as described in Sultana A. Lee J. Cuff Protoc Protein Sci, 79:19.25.1-19.25.26 (2015). Affinity of a binding agent may be determined using a soluble form of the binding agent or a membrane-tethered form, such as a chimeric antigen receptor (CAR) or T-cell receptor (TCR) fusion protein (TFP). Conversely, the pMHC complex may be tested in a soluble form or in its native, cell-membrane-bound state.

### A. Antibodies

In some embodiments, the binding agent is an antibody or antibody fragment. Suitable antibody fragments for practicing some embodiments of the disclosure include between one and three complementarity-determining region (CDRs) of an immunoglobulin light chain (referred to herein as “light chain”) and between one and three CDRs of an immunoglobulin heavy chain (referred to herein as “heavy chain”). Optionally, the binding agent comprises a variable region of a light chain, a variable region of a heavy chain, a light chain, or a heavy chain.

The identity of the amino acid residues in a particular antibody that make up a variable region or a CDR can be determined using methods well known in the art and include methods such as sequence variability as defined by Kabat et al. (See, e.g., Kabat et al., 1992 Sequences of Proteins of Immunological Interest, 5th ed., Public Health Service, NIH, Washington D.C), location of the structural loop regions as defined by Chothia et al. (see, e.g., Chothia et al., Nature 342:877-883 (1989), a compromise between Kabat and Chothia using Oxford Molecular's AbM antibody modeling software (now Accelrys®, see, Martin et al. Proc Natl Acad Sci USA. 86:9268 (1989); and world wide web site world-wide-web at bioinf-org.uk/abs), available complex crystal structures as defined by the contact definition (see MacCallum et al. J. Mol. Biol. 262:732-745 (1996)), the “conformational definition” (see, e.g., Makabe et al., Journal of Biological Chemistry, 283:1156-1166 (2008)), and the IMGT method (Lefranc M P, et al. IMGT unique numbering for immunoglobulin and T cell receptor variable domains and Ig superfamily V-like domains Dev Comp Immunol 27: 55-77 (2003)).

In embodiments, the binding agent is a functional antibody fragment comprising whole or essentially whole variable regions of both light and heavy chain, including but not limited to those defined as follows: (i) Fv, defined as a fragment consisting of the variable region of the light chain (VL) and the variable region of the heavy chain (VH) expressed as two chains; (ii) single chain variable fragment or single chain Fv (“scFv”), a genetically engineered single chain molecule including the variable region of the light chain and the variable region of the heavy chain, linked by a suitable polypeptide linker as a genetically fused single chain molecule; (iii) disulfide-stabilized Fv (“dsFv”), a genetically engineered antibody including the variable region of the light chain and the variable region of the heavy chain, linked by a genetically engineered disulfide bond; (iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CHI domains thereof; (v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule); (vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and (vii) single domain antibodies or nanobodies are composed of a single V_{H} or V_{L} domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988).

Antibody fragments according to some embodiments of the disclosure can be prepared by proteolytic hydrolysis of the antibody or by expression in E. coli or mammalian cells (e.g., Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein. See also Porter, R. R., Biochem J. 73:119-126 (1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In an embodiment in which the binding agent is an antibody, the heavy and light chains of an antibody of the disclosure may be full-length (e.g., an antibody can include at least one, and preferably two, complete heavy chains, and at least one, or two, complete light chains). In some embodiments, the antibody heavy chain constant region is chosen from, e.g., IgG1, IgG2, IgG3, IgG4, IgM, IgAQ1, IgA2, IgD, and IgE. In some embodiments, the immunoglobulin isotype is selected from IgG1, IgG2, IgG3, and IgG4, more particularly, IgG1 (e.g., human IgG1) or IgG4 (e.g., human IgG4). The choice of antibody type will depend on the immune effector function that the antibody is designed to elicit. In an embodiment, the binding agent elicits antibody dependent cellular cytotoxicity. In an embodiment, the binding agent elicits complement dependent cytotoxicity.

Bispecific configurations of antibodies are also contemplated herein. A bispecific monoclonal antibody (BsMAb, BsAb) is an artificial protein, or complex of proteins, that is composed of fragments of two different monoclonal antibodies and consequently binds to two different types of antigens. According to a specific embodiment the BsMAb is engineered to simultaneously bind to an effector cell (e.g., using a receptor like CD3) and a target like a tumor cell to be destroyed. Anti-CD3 antibodies known to the art and used for directing bispecific antibody engagement with CD3-positive effector cells include SP-34 (Pessano et al., EMBO J (1985) 4:337-344), OKT3 (Kung et al., Science (1979) 206: 347-349), UCHT1 (Beverley P C L, Callard R E Eur J Immunol (1981) 11:329), 12F6 (Wong J T and Colvin R B, J Immunol (1987) 139:1369-1374), and humanized and/or affinity engineered variants of all (e.g. Shalaby et al., J Exp Med (1992) 175:217-225). Other affinity scaffolds, such as VHH domains, may also be used to engineer CD3 binding (e.g. WO/2015/095412). Other configurations, such as tri-specific or tetra-specific antibodies, for example, are also contemplated.

B. Single-Chain Variable Fragment (scFv)

Fv fragments comprise an association of V_{H} and V_{L} chains. This association may be noncovalent, as described in Inbar et al. Proc Natl Acad. Sci. USA 69:2659-62 (1972). Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are described, for example, by Whitlow and Filpula, Methods 2:97-105 (1991); Bird et al., Science 242:423-426 (1988); Pack et al., Bio/Technology 11:1271-77 (1993); and U.S. Pat. No. 4,946,778. The resulting polypeptides can fold back on themselves to form antigen-binding monomers, or they can form multimers (e.g., dimers, trimers, or tetramers), depending on the length of a flexible linker between the two variable domains (Kortt et al., Prot Eng 10:423 (1997); Kortt et al, Biomol Eng 18:95-108 (2001)). By combining different V_{L} and V_{H}-comprising polypeptides, one can form multimeric scFvs that bind to different epitopes (Kriangkum et al., (2001) Biomol. Eng. 18:31-40). Techniques developed for the production of single chain antibodies include those described in U.S. Pat. No. 4,946,778; Bird, Science 242:423 (1988); Huston et al. Proc Natl Acad Sci USA 85:5879 (1988); Ward et al. Nature 334:544 (1989), de Graaf et al. Methods Mol Biol. 178:379-87 (2002). Single chain antibodies derived from binding provided herein include, but are not limited to, scFvs comprising one or more variable domain sequences, or one or more CDR sequences from one or more variable domain sequences, disclosed herein.

### C. Chimeric Antigen Receptor (CAR) and TCR Fusion Proteins (TFP)

Chimeric antigen receptors (CARs) are fusion proteins comprising antigen recognition moieties and T cell-activation domains. Exemplary CARs are provided by U.S. Pat. Nos. 8,399,645 and 7,638,325. Other exemplary recombinant receptors, including CARs, recombinant T-cell receptors (TCRs), TCR fusion proteins (TFPs), as well as methods for engineering and introducing the receptors into cells, include those described in Int'l Pat. Appl. Nos. WO2017/096329, WO2000/14257, WO2013/126726, WO2012/129514, WO2014031687, WO2013/166321, and WO2013/071154, WO2013/123061, and WO/2014055668; U.S. Pat. App. Nos. US2002131960, US2013287748, and US20130149337; U.S. Pat. Nos. 6,451,995, 7,446,190, 7,638,325, 8,252,592, 8,339,645, 8,398,282, 7,446,179, 6,410,319, 7,070,995, 7,265,209, 7,354,762, 7,446,191, 8,324,353, and 8,479,118; European Pat. App. No. EP2537416; and Sadelain et al. Cancer Discov. April 3(4): 388-398 (2013); Davila et al. PLoS ONE 8(4): e61338 (2013); Turtle et al. Curr. Opin. Immunol. October 24(5): 633-39 (2012); and Wu et al. Cancer, March 18(2): 160-75 (2012). In an embodiment, the binding agent is a TFP as described in U.S. patent Ser. No. 15/419,398.

### D. Amino Acid Substitutions

As discussed herein, minor variations in the amino acid sequences of the binding agents are contemplated as being encompassed by the present disclosure, providing that the variations in the amino acid sequence maintain at least 75%, more preferably at least 80%, 90%, 95%, and most preferably 99% sequence identity to the variable domains, or at least 50, 55, 60, 65, 70, 75, 80, 85, 90 or 95% sequence identity to single CDR regions for constructs disclosed here, so long as the binding affinity to the PHOX2B:HLA complex is functionally maintained. For example, the binding agent may comprise one or more amino acid substitutions relative to a CDR sequence provided herein. The binding agents may also comprise one or more amino acid substitutions in a framework region. In some embodiments, the binding agent may have no more than 2 amino acid substitutions in the CDR-L1, no more than 2 amino acid substitutions in the CDR-L2, no more than 3 amino acid substitutions in the CDR-L3, no more than 2 amino acid substitutions in the CDR-H1, no more than 2 amino acid substitutions in the CDR-H2, or no more than 4 amino acid substitutions in the CDR-H3, relative to any one or more of the CDR amino acid sequences provided herein. In some embodiments, the binding agent comprises an amino acid substitution in a framework region. For example, as a person skilled in the art would appreciate, routine site-directed or random mutagenesis techniques can be performed to alter the amino acid sequence of any one of the binding agents described herein in order to, for example, alter binding affinity (e.g., affinity maturation), reduce susceptibility to proteolysis or oxidation, or confer or modify other physicochemical or functional properties of the binding agents.

In some embodiments, the amino acid substitutions are conservative amino acid substitutions. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More particular families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Examples of conservative amino acid substitutions are provided below in Table 1.

TABLE 1
Exemplary conservative amino acid changes

| Original | | Preferred |
|---|---|---|
| amino acid | Exemplary substitutions | substitutions |
| Ala | Val; Leu; Ile | Val |
| Arg | Lys; Gln; Asn | Lys |
| Asn | Gln; His; Asp, Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Ala | Ala |
| His | Asn; Gln; Lys; Arg | Arg |
| Ile | Leu; Val; Met; Ala; Phe; Norleucine | Leu |
| Leu | Norleucine; Ile; Val; Met; Ala; Phe | Ile |
| Lys | Arg; Gln; Asn | Arg |
| Met | Leu; Phe; Ile | Leu |
| Phe | Trp; Leu; Val; Ile; Ala; Tyr | Tyr |
| Pro | Ala | Ala |
| Ser | Thr | Thr |
| Thr | Val; Ser | Ser |
| Trp | Tyr; Phe | Tyr |
| Tyr | Trp; Phe; Thr; Ser | Phe |
| Val | Ile; Leu; Met; Phe; Ala; Norleucine | Leu |

The present disclosure also contemplates non-conservative amino acid substitutions in a binding agent of the disclosure, provided that the binding agent is still capable of specifically binding to an HLA-A*24:02/PHOX2B complex, an HLA-A*23:01/PHOX2B complex, and other complexes listed in the table below.

TABLE 0

| | USA (Global) | | |
|---|---|---|---|
| Allele | Freq | | Mutations relative to A*24:02 |
| A2402 | 9.9% | (9.5%) | none |
| A2301 | 3.5% | (2.7%) | K144Q, H151R, Q156L |
| A2317 | 0% | (0.13%) | K144Q, H151R, Q156L |
| A2325 | 0% | (0.32%) | K144Q, H151R, Q156L, G175R |
| A2339 | 0% | (0.11%) | Q43R, K144Q, H151R, Q156L |
| A2404 | 0.01% | (0.22%) | E76A, R79G, I80T, A81L, L82R, R83G |
| A2407 | 0.43% | (0.22%) | H70Q |
| A24124 | 0% | (0.32%) | G56R |
| A24143 | 2.02% | (2.02%) | T163L |
| A24242 | 1.57% | (1.57%) | V28A |
| A24314 | 0.29% | (0.29%) | R111H |
| A24353 | 0.41% | (0.38%) | none |
| A2441 | 4.85% | (3.29%) | S105P, H114R, Y116D |
| A2451 | 0.76% | (0.76%) | M97I, F99Y, S105P |
| A2492 | 0% | (0.32%) | L95I, M97I, F99Y, S105P |
| A2403 | 0.29% | (0.23%) | D166E, G167W |
| A2433 | 0% | (0.69%) | D166E, G167W |
| A2487 | 0% | (0.6%) | Q156R |
| 18 alleles | 24.0% | (23.8%) | |

Confirmed non-binders to PHOX2B peptide: A*30:01, A*32:01, A*66:01, G*01:01
Confirmed non-binders to 10LH: A*29:02, A*31:08, A*32:07, B*08:01, B*14:02, C*07:02

In some embodiments, the amino acid substitutions are non-conservative amino acid substitutions. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Particular amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known, such as Bowie et al., Science 253:164 (1991) or (Am J Hum Genet., 2004). Thus, the foregoing examples demonstrate that those of skill in the art can recognize sequence motifs and structural conformations that may be used to define structural and functional domains in accordance with the antibodies described herein.

Particular amino acid substitutions are those which: (1) reduce susceptibility to proteolysis, (2) reduce susceptibility to oxidation, (3) alter binding affinities, and (4) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991); and Thornton et al. Nature 354:105 (1991).

Routine techniques can be used to introduce amino acid substitutions in CDRs to, for example, improve binding affinity. Such substitutions may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact the antigen, with the resulting variant being tested for binding affinity. Alternatively, or additionally, affinity maturation may be performed. Affinity maturation by constructing and reselecting from secondary libraries has been described, e.g., in Hoogenboom et al., Methods in Molecular Biology 178:1-37 (O'Brien et al., ed., Human Press, Totowa, NJ, (2001)). In some embodiments of affinity maturation, diversity is introduced into the variable region coding sequences chosen for maturation by any of a variety of methods (e.g., error-prone PCR, chain shuffling, or oligonucleotide-directed mutagenesis). A secondary library is then created. The library is then screened to identify any variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis, described below, or modelling. CDR-H3 and CDR-L3 in particular can be used for random mutagenesis and affinity maturation.

In certain embodiments, substitutions, insertions, or deletions may occur within one or more CDRs so long as such alterations do not substantially reduce the ability of the binding agent to bind an HLA-A*24:02/PHOX2B complex, an HLA-A*23:01/PHOX2B complex, or a complex listed in Table 0 above. In some embodiments, the binding agent comprising the amino acid substitutions binds to an HLA-A*24:02/PHOX2B complex, an HLA-A*23:01/PHOX2B complex or an HLA-A*24:02/PHOX2B complex, or an HLA-A*23:01/PHOX2B complex with a similar affinity to the binding agent without the substitutions. Such substitutions may, for example, be outside of antigen-contacting residues in the CDRs. In some embodiments, the binding agent comprising the amino acid substitutions binds to an HLA-A*24:02/PHOX2B complex, or an HLA-A*23:01/PHOX2B complex with a higher affinity than the binding agent without the substitutions. In some embodiments, the binding agent comprising the amino acid substitutions binds to an HLA-A*24:02/PHOX2B complex, or an HLA-A*23:01/PHOX2B complex with a lower affinity than the binding agent without the substitutions. In certain embodiments, each CDR either is unaltered, or contains no more than one, two, three, or four amino acid substitutions. In some embodiments, the substitutions are conservative substitutions.

A useful method for identification of residues or regions of a binding agent that may be targeted for mutagenesis is called “alanine scanning mutagenesis” as described by Cunningham, Science 244:1081-1085 (1989). In this method, a residue or group of target residues (e.g., charged residues such as Arg, Asp, His, Lys, and Glu) are identified and replaced by a neutral amino acid such as alanine to determine whether the interaction of the binding agent with its antigen is affected. Further substitutions may be introduced at the amino acid locations demonstrating functional sensitivity to the initial substitutions. Alternatively, or additionally, a crystal structure of an antigen-binding agent complex can be used to identify contact points between the binding agent and antigen. Such contact residues and neighbouring residues may be targeted or eliminated as candidates for substitution. Variants may be screened to determine whether they contain the desired properties.

### III. NUCLEIC ACIDS

According to an aspect of the disclosure there is also provided an isolated polynucleotide comprising a nucleic acid sequence encoding the binding agent as described herein. Also provided is an expression vector, comprising the polynucleotide operably linked to a cis-acting regulatory element. The expression vector of some embodiments of the disclosure includes additional sequences which render this vector suitable for replication and integration in prokaryotes, eukaryotes, or preferably both (e.g., as a shuttle vector). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the disclosure includes a signal sequence for secretion or presentation of the binding agent from a host cell in which it is placed. Preferably the signal sequence for this purpose is a mammalian signal sequence.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. Preferably, the promoter utilized by the expression vector is active in the specific cell population transformed. Examples of cell type-specific and/or tissue-specific promoters include promoters such as albumin that is liver specific (Pinkert et al. Genes Dev. 1:268-277 (1987)), lymphoid specific promoters (Calame et al. Adv. Immunol. 43:235-275 (1988)); in particular promoters of T-cell receptors (Winoto et al. EMBO J. 8:729-733 (1989)) and immunoglobulins; (Banerji et al. Cell 33:729-740 (1983)), neuron-specific promoters such as the neurofilament promoter (Byrne et al. Proc. Natl. Acad. Sci. USA 86:5473-5477 (1989)), pancreas-specific promoters (Edlunch et al. Science 230:912-916 (1985)) or mammary gland-specific promoters such as the milk whey promoter (U.S. Pat. No. 4,873,316 and European Application Publication No. EP0264166). In the construction of the expression vector, the promoter is preferably positioned approximately the same distance from the heterologous transcription start site as it is from the transcription start site in its natural setting. As is known in the art; however, some variation in this distance can be accommodated without loss of promoter function.

Enhancer elements can stimulate transcription up to 1,000-fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the disclosure include those derived from polyoma virus, human or murine cytomegalovirus (CMV), the long-term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983.

Polyadenylation sequences can also be added to the expression vector in order to increase the efficiency of TCRL mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the disclosure include those derived from SV40.

In addition to the elements already described the expression vector of some embodiments of the disclosure may contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein. Such cells are typically selected for high expression of recombinant proteins (e.g., bacterial, plant or eukaryotic cells e.g., CHO, HEK-293 cells), but may also be host cells having a specific immune effector activity (e.g., T cells or NK cells) when for instance the CDRs of the TCRL are implanted in a T Cell Receptor or CAR transduced in said cells which are used in adoptive cell therapy.

### IV. DIAGNOSTIC APPLICATIONS

The high specificity of the binding agent renders it particularly suitable for diagnostic and therapeutic applications. According to an aspect of the present disclosure, there is provided a method of detecting a cell presenting an HLA-restricted peptide antigen of interest. The method comprises contacting the cell with the binding agent (e.g., antibody) of the present disclosure having specificity to the HLA-restricted peptide antigen of interest. The contacting is effected under conditions which allow immunocomplex formation, wherein a presence of the immunocomplex or the level thereof is indicative of the cell presenting the HLA-restricted peptide antigen of interest.

The term “detecting,” as used herein, refers to the act of detecting, perceiving, uncovering, exposing, visualizing or identifying a cell. The precise method of detecting is dependent on the detectable moiety to which the antibody is attached.

Single cells may be used for detection as well as a plurality of cells. For instance, the cells may be from any biological sample such as cell lines, primary cells (e.g., tumor cultures), and cellular samples (e.g., surgical biopsies including incisional or excisional biopsy, fine needle aspirates and the like). Methods of biopsy retrieval are well known in the art. The above-mentioned detection method can be harnessed to the diagnosis of diseases (such as cancer) which are characterized by above normal presentation or different tissue distribution of the HLA-peptide complex.

As used herein the term “diagnosing” refers to classifying a disease, determining a severity of a disease (grade or stage), monitoring progression, forecasting an outcome of the disease and/or prospects of recovery.

The subject may be a healthy subject (e.g., human) undergoing a routine well-being check-up. Alternatively, the subject may be at risk of the disease. The method may be used to monitor treatment efficacy. The binding agent may comprise, that is, be attached to, a detectable moiety. Alternatively or additionally, the binding agent (or a complex comprising same) may be identified indirectly such as by using a secondary antibody. The contacting may be effected in vitro (i.e., in a cell line, primary cells), ex vivo, or in vivo.

### V. PHARMACEUTICAL COMPOSITIONS, FORMULATIONS AND DOSAGES

Pharmaceutical compositions according to the present disclosure, and for use in accordance with the present disclosure, may comprise, in addition to the active ingredient, (i.e., the binding agent), a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g., cutaneous, subcutaneous, or intravenous.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

In one embodiment, the composition is a pharmaceutical composition (e.g., formulation, preparation, medicament) comprising a binding agent, as described herein, and a pharmaceutically acceptable carrier, diluent, or excipient.

In one embodiment, the composition is a pharmaceutical composition comprising at least one binding agent, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilizers, solubilizers, surfactants (e.g., wetting agents), masking agents, coloring agents, flavoring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash) Synapse Information Resources, Inc., Endicott, New York, USA (2001), Remington's Pharmaceutical Sciences, 20th edition, pub. Lippincott, Williams & Wilkins, (2000); and Handbook of Pharmaceutical Excipients, 2nd edition (1994).

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as antioxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It will be appreciated by one of skill in the art that appropriate dosages of the binding agent, and compositions comprising the binding agent, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of binding agent and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In some embodiments, the binding agent or composition containing the same is administered once per week for a therapeutically effective period of time. In some embodiments, the binding agent or composition containing the same is administered once per day for a therapeutically effective period of time. In some embodiments, the binding agent or composition containing the same is administered once per month for a therapeutically effective period of time. In some embodiments, the binding agent or composition containing the same is administered once per year for a therapeutically effective period of time.

In general, a suitable dose of the binding agent is in the range of about 100 ng to about 25 mg (more typically about 1 g to about 10 mg) per kilogram body weight of the subject per day. Where the composition comprises a salt, an ester, an amide, a prodrug, or the like, the amount administered is calculated on the basis of the parent compound and so the actual weight to be used is increased proportionately.

### VI. TREATMENT

The binding agents of the disclosure (e.g., antibodies, CARs) are especially useful for the treatment of cancer. In particular embodiments, the cancer is characterized by expression of PHOX2B. Types of cancers to be treated with the binding agents of the disclosure include, but are not limited to, hematological cancers, solid tumors, and non-solid tumors. Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases). Adult tumors/cancers and pediatric tumors/cancers are also included.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically effective amount,” as used herein, pertains to that amount of binding agent, or a material such as an antibody-drug conjugate, composition or dosage form comprising an active binding agent, which is effective for producing some desired therapeutic effect when administered in accordance with a desired treatment regimen.

In some embodiments, the treatment reduces or inhibits tumor growth for at least 6, 12, 24, 36, or 48 months. In some embodiments, the treatment enhances an immune response against the tumor.

The subject/patient may be an animal or any species of mammal, including, without limitation, a horse, a dog, a cat, a pig, or a primate. In a particular embodiment, the subject/patient is a human.

### VII. SCREEN FOR CROSS-REACTIVITY OF A CANDIDATE PEPTIDE:HLA TARGETED THERAPEUTIC AGAINST OFF-TARGET HLAS

As used herein, the terms “native,” “native human leukocyte antigen,” and “native HLA” refer to an HLA or fragment thereof that maintains the structural and antigenic property of the extracellular portion of an HLA in its native state.

As used herein, the terms “native class I human leukocyte antigen,” and “native class I HLA” refer to a class I human leukocyte antigen or fragment thereof that maintains the structural and antigenic integrity of the extracellular portion of a class I HLA in its native state, including comprising a β2 microglobulin domain noncovalently bound to a heavy chain or fragment thereof. In some embodiments, native class I HLAs are capable of binding to W6/32 and BIH antibodies (One Lambda, Inc.).

As used herein, the terms “native class II human leukocyte antigen,” and “native class II HLA” refer to a class II human leukocyte antigen or fragment thereof that maintains the structural and antigenic integrity of the extracellular portion of a class II HLA in its native state, including comprising a heterodimer that comprises two glycosylated polypeptide chains noncovalently bound to each other. In some embodiments, native class II HLAs are capable of binding to HB-145 (directed to HLA-DP, HLA-DQ, HLA-DR) and HB-180 (directed to HLA-DQ and HLA DR) antibodies (American Type Cultural Collection (ATCC)).

As used herein, the terms “denatured,” “denatured human leukocyte antigen,” and “denatured HLA” refer to an HLA comprising an extracellular domain that is not in a native confirmation.

As used herein, the terms “denatured class I human leukocyte antigen,” and “denatured class I HLA” refer to class I human leukocyte antigen or fragment thereof that lacks a β2 microglobulin domain. In some embodiments, denatured class I HLAs are capable of binding to HC10 (One Lambda, Inc,) and HB296 antibodies (ATCC).

As used herein, the terms “denatured class II human leukocyte antigen” and “denatured class II HLA” refer to a class II human leukocyte antigen or fragment thereof that is in a monomeric confirmation. In some embodiments, denatured class II HLAs are capable of binding HB-298 (directed to HLA-DR a chain) antibody (ATCC).

As used herein, the term “solid substrate” refers to any solid substrate that is capable of binding HLAs and is compatible with the methods provided herein. Examples of solid substrates include a plurality of beads, a plurality of microbeads, a plurality of microparticles, a plurality of microspheres, a well, a membrane, a polymer, a filter, a microarray and combinations thereof.

As used herein, the term “same HLA allele” refers to two or more HLA molecules or fragments thereof that share similar structure and antigenic properties and are derived from the same HLA gene loci and alleles.

As used herein, the term “different HLA allele” refers to HLA molecules or fragments thereof that possess different structure and antigenic properties and are derived from different HLA gene loci and alleles.

Provided herein is a composition comprising at least 90% native HLAs and at most 10% denatured HLAs. In certain embodiments, the composition can be used for the detection of antibodies to native HLAs. Detection of cross-reactivity of a candidate peptide:HLA targeted therapeutic against off-target HLAs can be useful, for example, in identifying and mitigating cross-reactivity risks during preclinical development of peptide:HLA-targeted therapeutics.

The composition can be made by any technique apparent to one of skill in the art, including the methods described herein. Each element of the composition is discussed in further detail below.

In some embodiments, the composition provided herein comprises at least 90% native HLAs and at most 10% denatured HLAs. In some embodiments, the HLAs are linked to a solid support. In other embodiments, the HLAs are in solution.

Native class I HLAs are 57 kDa glycoproteins that are present on most nucleated human cells. Native class I HLAs typically comprise a 45 kDa polypeptide heavy chain bound to a light chain that comprises a 12 kDa β2 microglobulin domain. In certain embodiments, the heavy chain is noncovalently bound to the light chain. The heavy chain typically comprises three subunits, a transmembrane domain, and a cytoplasmic tail. The α1 and α2 subunits form a binding groove for peptide ligand binding. A denatured class I HLA, in contrast, lacks a β2 microglobulin domain.

The class I HLA heavy chain is encoded by one of three major genes (HLA-A, HLA-B and HLA-C) or one of three minor genes (HLA-E, HLA-F and HLA-G). Allelic variation within each of these gene loci helps contribute to the polymorphism exhibited by class I HLA. A particular class I HLA can be categorized by the gene locus and the particular allele from which the class I HLA is expressed.

Native class IT HLAs are polymorphic 61 kDa heterodimeric proteins that are present on the surface of specialized antigen presenting cells (e.g., B lymphocytes, dendritic cells, and macrophages). Class II HLAs are divided into three subclasses: HLA-DP, HLA-DQ and HLA-DR. A native class II HLA typically comprises an α chain and β chain bound to each other. In certain embodiments, the α chain and β chain are noncovalently bound to each other. In contrast, denatured class II HLAs are monomeric proteins.

Each chain of a class II HLA comprises an extracellular domain, a transmembrane domain and a cytoplasmic tail. There are six major class II HLA genes (HLA-DPA1, HLA-PB1, HLA-DQA1, HLA-DQB1, HLA-DRA and HLADRB1), each gene encoding either an α or β chain. Similar to the class I HLA genes, each class II HLA gene comprises many alleles. Class I and class II HLAs can be made using any technique known to those of skill in the art including recombinant DNA techniques as described in Pei et al., Transplantation 75(1): 43-49 (2003).

In some embodiments, the composition comprises a substantial amount of native HLAs. In some embodiments, at least 90% of the HLAs are native and at most 10% of the HLAs are denatured. In some embodiments, at least 95% of the HLAs are native and at most 5% of the HLAs are denatured. In some embodiments, at least 99% of the HLAs are native and at most 1% of the HLAs are denatured. In some embodiments, at least 99.5% of the HLAs are native and at most 0.5% of the HLAs are denatured. Techniques for making these compositions are described below.

In some embodiments, the composition comprises HLAs selected from the group consisting of class I HLAs, class II HLAs and combinations thereof. In some embodiments, the composition comprises class I HLAs. In some embodiments, the composition comprises class II HLAs. In some embodiments, the composition comprises a combination of class I and class II HLAs.

The composition can comprise full length native HLAs or fragments thereof. In some embodiments, the composition comprises full length native class I HLAs, each class I HLA comprising a heavy chain noncovalently bound to a β2 microglobulin chain. In some embodiments, the composition comprises full length class II HLAs, each class II HLA comprising an α chain noncovalently bound to a β chain.

In some embodiments, each HLA comprises a modification such as a deletion, addition or amino acid substitution that does not disrupt the structural and antigenic integrity of the native HLA extracellular domain. In some embodiments, each HLA is a class I HLA that comprises an extracellular domain of a native class I HLA. In some embodiments, each HLA is a class I HLA that comprises an extracellular domain and a fragment of a transmembrane domain of a native class I HLA. In some embodiments, each HLA is a class I HLA that comprises an extracellular domain and a transmembrane domain of a native class I HLA. In some embodiments, each HLA is a class I HLA that comprises an extracellular domain, a transmembrane domain and a fragment of a cytoplasmic tail of a native class I HLA.

In some embodiments, each HLA is a class II HLA that comprises an extracellular domain of a native class II HLA. In some embodiments, each HLA is a class II HLA that comprises an extracellular domain and a fragment of a transmembrane domain of a native class II HLA. In some embodiments, each HLA is a class II HLA that comprises an extracellular domain and a transmembrane domain of a native class II HLA. In some embodiments, each HLA is a class II HLA that comprises an extracellular domain, a transmembrane domain and a fragment of a cytoplasmic tail of a native class II HLA.

The composition provided herein can comprise HLAs of the same allele or to two or more different alleles. HLAs that are of the same allele share the same structure and antigenic properties and are derived from the same HLA loci and alleles.

In some embodiments, at least 90% of the HLAs are of the same allele. In some embodiments, at least 95% of the HLAs are of the same allele. In other embodiments, at least 99% of the HLAs are of the same allele.

In some embodiments, at least 90% of the HLAs are of the same allele and comprise an HLA-A class I HLA. In some embodiments, at least 90% of the HLAs are of the same allele and comprise an HLA-B class I HLA. In some embodiments, at least 90% of the HLAs are of the same allele and comprise an HLA-C class I HLA.

In some embodiments, at least 90% of the HLAs are of the same allele and comprise an HLA-DP class II HLA. In some embodiments, at least 90% of the HLAs are of the same allele and comprise an HLA-DQ class II HLA. In some embodiments, at least 90% of the HLAs are of the same allele and comprise an HLA-DR class II HLA.

In certain embodiments, the HLAs provided herein are linked to a solid substrate. Native HLAs linked to the solid substrate allow the composition to bind antibodies to native HLAs. The bound antibodies to native HLAs can be detected using any technique known to those of skill in the art.

In certain embodiments, a substantial amount of the HLAs linked to the solid substrate are native. As used herein, a “substantial amount” can be any amount that allows for the binding and detection of antibodies to native HLAs without significant binding of antibodies specific for denatured HLAs. This amount can be expressed as a percentage of the total number of native HLAs to total number of HLAs linked to the solid substrate. In some embodiments, at least 75% of the HLAs are native. In some embodiments, at least 80% of the HLAs are native. In some embodiments, at least 85% of the HLAs are native. In some embodiments, at least 90% of the HLAs are native. In some embodiments, at least 95% of the HLAs are native. In some embodiments, at least 99% of the HLAs are native. In some embodiments, at least 99.5% of the HLAs are native.

HLAs can be linked to the solid substrate by any technique known to those of skill in the art. Further, HLAs can be directly or indirectly linked to the solid substrate. In some embodiments. HLAs are directly linked to the solid substrate. In some embodiments, HLAs are directly linked to the solid substrate by absorption, chemical coupling or by chemical linkage through a tail element added to the HLA. In certain embodiments. HLAs are directly linked to the substrate by passive absorption. Cantarero et al., Anal. Biochem., 105: 373-382 (1980). In some embodiments, HLAs are indirectly linked to the solid substrate by a linking moiety. In some embodiments, the linking moiety is selected from the group consisting of an antibody, a lectin, a CD8 molecule, a CD4 molecule, a T cell receptor and fragments thereof. In some embodiments, the linking moiety is a bifunctional cross-linker. Useful bifunctional cross-linkers are known to those of skill in the art.

The solid substrate can be made of any material known to those of skill in the art that is able to link to HLAs. Well known materials for solid substrates include glass, polystyrene, polypropylene, polyethylene, dextran, nylon, amylases, natural and modified celluloses, polyacrylamides, polysulfone, hydrogel, polyvinyl and magnetite. In some embodiments, the solid substrate comprises a material selected from the group consisting of silica, gold, latex, polystyrene, polyethylene, polysulfone, hydrogel, polyvinyl chloride, glass, and combinations thereof.

Further, the solid substrate can have any structural configuration deemed suitable by those of skill in the art. Solid substrates can comprise a plurality of beads, a plurality of microbeads, a plurality of microparticles, a plurality of microspheres, a well, a membrane, a polymer, a filter, a microarray and combinations thereof.

In some embodiments, the solid substrate is a plurality of microbeads. Useful microbeads are commercially available from sources such as Luminex, Inc., Invitrogen Corp., Polysciences, Inc. and Bangs Laboratories, Inc. to name a few. In certain embodiments, the microbeads are 2 to 8 m in diameter. In certain embodiments, the microbeads are 4 to 6 m in diameter.

In some embodiments, the solid substrate is a plurality of microparticles. In some embodiments, the microparticles are nanocrystals or quantum dots.

The solid substrate can also comprise a detectable label or any other identifying characteristic that can allow for the identification, separation and classification of antibodies bound to the HLAs. For example, the substrate can be a plurality of microbeads, each labeled with a fluorophore that allows the microbeads to be sorted using flow cytometry. Detectable labels can include fluorescent dyes, radioactive labels, magnetic labels, bar codes and combinations thereof. In certain embodiments, the detectable label is a fluorescent dye. In certain embodiments, the detectable label is a radioactive label. In certain embodiments, the detectable label is a bar code. In certain embodiments, the substrate comprises a detectable label selected from the group consisting of a fluorescent dye, a radioactive label, a magnetic label, a bar code and combinations thereof.

In another aspect provided herein are panels comprising a plurality of solid substrates, wherein each solid substrate of the plurality is linked to HLAs, wherein at least 90% of the HLAs linked are native and at most 10% of the HLAs are denatured, wherein at least 90% of the HLAs linked to a particular solid substrate of the plurality are of the same allele, and wherein each solid substrate of the plurality is linked to a different HLA with respect to the other solid substrates of the plurality. The panels can be linked to any of the HLAs provided herein. Panels advantageously allow for the detection of multiple peptide:HLA-targeted therapeutic to one or more native class I HLAs at a time.

In some embodiments, the plurality comprises at least 2 or more solid substrates. In some embodiments, the plurality comprises at least 4 or more solid substrates. In some embodiments, the plurality comprises at least 8 or more solid substrates. In some embodiments, the plurality comprises at least 16 or more solid substrates. In some embodiments, the plurality comprises at least 32 or more solid substrates. In some embodiments, the plurality comprises at least 64 or more solid substrates. In some embodiments, the plurality comprises at least 128 or more solid substrates. In some embodiments, the plurality comprises at least 256 or more solid substrates.

In some embodiments, each solid substrate of the plurality is linked to HLA-A class I HLAs. In some embodiments, each solid substrate of the plurality is linked to HLA-B class I HLAs. In some embodiments, each solid substrate of the plurality is linked to HLA-C class I HLAs.

In some embodiments, each solid substrate of the plurality is linked to HLA-DP class II HLAs. In some embodiments, each solid substrate of the plurality is linked to HLA-DQ class II HLAs. In some embodiments, each solid substrate of the plurality is to HLA-DR class II HLAs.

In some embodiments, the panel comprises a plurality of substrates linked to HLAs selected from the group consisting of HLA-A, HLA-B. HLA-C, HLA-DP, HLA-DQ, HLA-DR and combinations thereof.

In some embodiments, at least 90% of the HLAs linked to a particular solid substrate are of the same allele. In some embodiments, at least 95% of the HLAs linked to a particular solid substrate are of the same allele. In some embodiments, at least 99% of the HLAs linked to a particular solid substrate are of the same allele. In some embodiments, at least 99.5% of the HLAs linked to a particular solid substrate are of the same allele.

### VIII. EXAMPLES

The following examples are included to demonstrate particular embodiments. It should be appreciated by those of skill in the art that the techniques disclosed in the examples that follow represent techniques discovered by the inventor to function well in the practice of embodiments, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Screening Methods

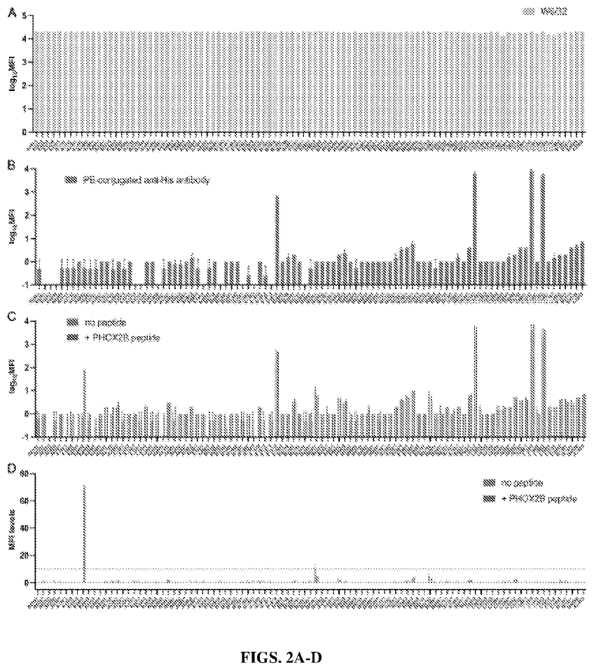

To test the levels of peptide-loaded MHC-I molecules on the SABs, the inventors used a PE-conjugated W6/32 antibody (Biolegend, 311406) mixed with 4 μL of the LABScreen single antigen HLA-I bead suspension (OneLambda, Inc., CA, USA) in a 96-well plate. The samples were incubated for 30 min, 550 rpm at room temperature (RT), washed four times in Wash Buffer (OneLambda, Inc., CA, USA) to remove excess of antibody and resuspended in phosphate-buffered saline (PBS), pH 7.2 (FIG. 2A). As a negative control experiment, the inventors used the same secondary antibody and incubated the beads for 30 min, 550 rpm at RT followed by four washes and detected the levels of background staining (FIG. 2B). To screen for scFv cross-reactive interactions, 28 nM of soluble protein were mixed with 4 μL of beads that were pre-incubated over weekend with either PBS buffer or with 100 molar excess of PHOX2B peptide (2.8 μM), under agitation at 4° C. Samples were incubated for 1 hour at 550 rpm, RT, washed three times followed by addition of a PE-conjugated anti-His tag antibody (Biolegend, 362603) for 30 min, 550 rpm at RT. After four washes in Wash Buffer, the levels of bound 10LH were detected (FIG. 2C). In all cases, the levels of fluorescence intensity were measured using the Luminex 100 Liquid Array Analyzer System and the results were analyzed in GraphPad Prism v9. The final plot was created by subtracting the Mean Fluorescence Intensity (MFI) of the negative control (b) from the binding levels observed upon scFv incubation with or without PHOX2B peptide addition (a) (FIG. 2D). Errors were propagated by the equation

$(δa)!+(δb)!

where δ is the error associated with each measurement from n=2 replicates. In the cases where the difference was negative, the inventors assumed 0 binding levels.

### Example 2—Results

The development of Chimeric Antigen Receptor (CAR) T cell therapies targeting peptide:HLA complexes containing fragments of intracellular oncoproteins that are expressed in tumors, is an emerging approach for the development of targeted cancer therapies to treat solid tumors⁸⁹. pHLA complexes comprise the most polymorphic proteins in the human proteome with more than 35,000 different HLA allotypes identified to date (Barker et al., 2022) which poses a challenge in the development of targeted therapies. An important bottleneck in the development of CAR-T and TCR-T therapies targeting specific peptide:HLA complexes displayed in tumors, is the development of binders that interact with a distinct peptide:HLA but don't cross-react with other, off-target peptide:HLA complexes. In light of the lethal toxicities arising from such cross-reactivities seen in recent TCR-T clinical trials (Linette et al., 2013), there is an unmet need for high-throughput assays that can probe binding of candidate therapeutics (TCRs, CARs, or any other pHLA targeting modality) against a panel of common HLA allotypes.

Here, the inventors have adapted a common assay used to evaluate the presence of HLA-specific antibodies in the sera of solid organ transplant recipients for histocompatibility matching, namely the Single Antigen Beads (SABs) (Pei et al., 1998; Pei et al., 1999), to evaluate cross-reactivity of a candidate peptide:HLA targeted therapeutic against off-target HLAs. This assay is routinely used in solid organ transplantation to evaluate the presence of alloreactive antibodies against HLAs present in sera. It has been previously adapted to study interactions between the molecular chaperone TAPBPR and different HLA allotypes (Ilca et al., 2019) and here, the inventors further adapt this method to screen for cross-reactive interactions with engineered peptide-specific single-chain antibody variable fragment (scFv) binders (Maus et al., 2016). In addition, the inventors have further modified the assay to evaluate the peptide-dependence of interactions with pHLAs, by incubating the beads with a target peptide, allowing exchange to occur. As proof of concept, the inventors used the single-chain antibody fragment 10LH which targets the neuroblastoma peptide PHOX2B (QYNPIRTTF) normally presented by HLA-A*24:02⁸⁹. These results demonstrate that 10LH does not cross-react with a panel of 97 different HLA allotypes presenting a broad variety of high-affinity peptides, while it is able to bind HLA-A*24:02 upon incubation with excess of the target peptide. These results highlight the use of an adapted SAB assay to identify and mitigate cross-reactivity risks during preclinical development of peptide:HLA-targeted therapeutics.

### Example 3—Development of PHOX2B 10LH

ReD library panning. The Ruby scFv library (>10¹¹ diversity) was constructed using human germline IGLV1-51, IGLV3-1 and IGLV6-57 scaffolds paired with the IGHV3-23 scaffold, as described by Beasley et al. (Beasley et al., 2015), with fully synthetic amino acid diversity in both V_{L} and V_{H} CDR3 loops.

The Ruby scFv library, and its combination and use with the Retained Display platform for antibody screening, are described by WO/2011/075761 (Protein display), WO/2013/023251 (Soluble polypeptides), and WO/2013/000023 (Method of Protein display).

The Ruby library was panned for two rounds using PHOX2B (43-51) A*24:02 MHC complex bound to MyOne Streptavidin C1 Dynabeads (ThermoFisher, Cat: 65002). Panned library output were transferred into the ReD cell-display platform (Beasley et al., 2015) and cells were permeabilized using 0.5% n-octyl β-d-thioglucopyranoside (Anatrace, Cat: 0314) and labeled using recombinant PHOX2B pMHC complex ligated to fluorophores excitable by 405 nm and 488 nm lasers. Cells that were positive for target binding were isolated using the FACSMelody sorter (Becton-Dickinson).

After two rounds of positive selection for binding to PHOX2B A*24:02 MHC complex two further FACS rounds were conducted using counter-labelled A*24:02 MHC complexes with unrelated peptides. After four rounds of FACS individual colonies were picked and grown in 96-well plates before scFv induction, cell permeabilization, and PHOX2B MHC labelling and detection by CytoFLEX (Beckman Coulter).

Clones that were identified as binding specifically to the PHOX2B (43-51) MHC complex were sequenced and unique scFvs were expressed as fusions to the AviTag™ biotinylation motif in E. coli. Biotinylated scFv protein was released via permeabilization with 0.5% n-octyl β-d-thioglucopyranoside and purified to ~90% purity on Nickel NTA agarose resin (ABT, Cat: 6BCL-NTANi).

Binding kinetics. Affinity measurements were performed using a BLItz™ system (ForteBio, USA) and analyzed using the BLItz Pro™ software. Streptavidin biosensors (ForteBio, Cat: 18-5019) were loaded with AviTag™-biotinylated scFv, blocked with biotin, washed in PBS, and then associated with pMHC ligand in PBS.

Steady-state binding assay. An equilibrium binding assay to target pMHCs was also established using MyOne Streptavidin C1 Dynabeads. Briefly, 50 micrograms of Streptavidin C1 Dynabeads were incubated with excess biotinylated scFv before being blocked with free biotin and washed in PBS. Fluorophore-labelled pMHC complex was added to a concentration of 3.5 nM and incubated for 1 hour at 4° C. followed by 10 minutes at 25° C. Binding of the free MHC complex to the beads was quantitated by the CytoFLEX at 488 nm (ex)/525 nm (em). Binding was normalized to beads without scFv and with unrelated control MHC complex.

This bead-binding assay was used to quantitate the binding of scFv to MHC complexes with alanine-scan substitutions of the PHOX2B peptides as well as to a plate of 95 unrelated 9-mer peptide A*24:02 MHC complexes and the degree of cross-reactivity of binding of MHC complexes with peptides identified as having high homology to the PHOX2B peptide by eXpitope 2.0.

Viral production and transduction of Jurkat and primary T cells. Retrovirus for transduction of Jurkats and primary CD4/8 T cells was produced using Platinum-A (Plat-A) cells, a retroviral packaging cell line. Cells were plated in 6-well plates at 7×10⁵ cells/well and transfected with 2.5 μg of the appropriate TCR or CAR construct in the retroviral vector pMP71 using Lipofectamine 3000 (Life Technologies, Invitrogen). After 24 hours, medium was replaced with IMDM-10% FBS or AIM-V-10% FBS for Jurkat cells or primary cells, respectively. Supernatants were harvested and filtered with 0.2 mM filters after 24 hours incubation.

A second-generation lentiviral system was used to produce replication-deficient lentivirus. The day preceding transfection, 15 million HEK 293T cells were plated in a 15-cm dish. On the day of transfection, 80 μL Lipofectamine 3000 (Life Technologies, Invitrogen) was added to 3.5 mL room-temperature Opti-MEM medium (Gibco). Concurrently, 80 μL P3000 reagent (Thermo Fisher Scientific), 12 μg psPAX2 (Gag/Pol), 6.5 μg pMD2.6 (VSV-G envelope), and a matching molar quantity of transfer plasmid were added to 3.5 mL room-temperature Opti-MEM medium. Virus supernatant was collected after 24 and 48 hours later and briefly centrifuged at 300 g and passed through a 0.45 μM syringe.

Jurkat cells were plated in 6-well plates pre-treated with 1 mL well/Retronectin (20 mg/mL, Takara Bio. Inc.,) at 1×10⁶ cells/well and spinoculated with 2 mL of retroviral supernatant at 800 xg for 30 min at RT. After 24 hours, cells were harvested, and grown in IMDM-10% FBS.

Primary T cells were thawed and activated in culture for 3 days in the presence of 100 U/ml IL-2 and anti-CD3/CD28 beads (Dynabeads, Human T-Activator CD3/CD28, Life Technologies) at a 3:1 bead:T cell ratio. On days 4 and 5, activated cells were plated in 6-well plates pre-treated with 1 mL well/Retronectin (20 mg/mL, Takara Bio. Inc.) at 1×10⁶ cells/well and spinoculated with 2 mL of retroviral supernatant at 2400 rpm for 2 hours at 32° C. On day 6, cells were harvested and washed, beads were magnetically removed, and cells were expanded in AIM-V-10% FBS supplemented with 25 U/ml IL-2.

Primary human T cells were thawed and activated in culture for 1 day in the presence of 5 ng/ml recombinant IL-7, 5 ng/ml recombinant IL-15, and anti-CD3/CD28 beads (Dynabeads, Human T-Activator CD3/CD28, Life Technologies) at a 3:1 bead:T cell ratio in G-Rex system vessels (Wilson Wolf). On day 2, thawed lentiviral vector was added to cultured T cells with 10 μg/mL Polybrene (Millipore Sigma), and 24 hours later vessels were filled with complete AIM-V medium supplemented with indicated concentrations of IL-7 and IL-15. On day 10, cells were harvested and washed. Activation beads were magnetically removed, and cell viability was determined before freezing.

Human neuroblastoma cell lines were plated in 6-cm dishes, and 2 mL of thawed lentiviral vector produced with transfer plasmid pLenti-CMV-eGFP-Puro (Addgene plasmid #17448) was added with 10 μg/mL Polybrene (Millipore Sigma). Cells were selected for eGFP expression using flow-assisted cell sorting (BD FACSJazz, BD Biosciences) followed by 10 μg/mL puromycin selection.

Selective Cross-Reactive Antigen Presentation (sCRAP) prediction. Tumor antigens were compared against the entire normal human proteome on the matched HLA (85,915,364 total normal peptides among HLA 84 HLAs). Each residue in the same position of the tumor and human peptides was assigned a score for perfect match, similar amino acid classification, or different polarity, scoring five, two, or negative two respectively. Similarity scores were calculated based on amino acid classification and hydrophobicity was determined using residues one and three through eight and excluding MHC anchor residues. Next, the maximum normal tissue RPKM values were identified from 1643 normal tissues in GTEx. Normal peptides were compared to a database of normal tissue immunopeptidomes (Shao et al., 2020). The overall cross-reactivity score for each normal peptide was then calculated using the following equation:

$$\frac{{\sum }_{i = 3}^{n} P i}{b * E max}$$

where n is the peptide length, P is the score of each amino acid of the normal peptide as compared to the tumor antigen, b is the pMHC binding affinity of the normal peptide, and Eₘₐₓ is the maximum normal tissue expression. The algorithm is available at marisshiny.research.chop.edu/sCRAP.

Tetramer/dextramer staining and flow cytometric analysis. Surface expression and binding of CAR-transduced Jurkat cells and primary T cells was measured by staining with PE- or APC-conjugated dextramers carrying NB antigen peptide-MHC (Immudex). Cells were harvested from culture, washed with 2 ml PBS at 800 xg for 5 min, incubated with 1 μl dextramer for 10 min in the dark, washed again, and resuspended in 300 ul PBS for analysis. Typically, 5×10⁵ cells were used for staining, and analyzed on a BD LSR II (BD Biosciences) or an Attune Acoustic Focusing Cytometer (Applied Biosystems, Life Technologies).

Cross-reactivity pMHC screen. Potential cross-reactive peptides (GenScript) were suspended at a 200 μM working concentration. For each test, 0.5 μL of peptide was added to 5 μL HLA-A*24:02 empty loadable tetramer (Tetramer Shop) before incubating on ice for 30 minutes, or using TAPBR peptide exchange as previously described (Overall et al., 2020). Following preparation, pMHC tetramers were used to stain cells (described above).

Incucyte Cytotoxicity Assay. 0.5×10⁵ tumor cell targets were co-incubated with varying ratios of transduced primary cells (5×10⁵, 2.5×10⁵, 1×10⁵, 0.5×10⁵, and 2.5×10⁴ for 10:1, 5:1, 2:1, 1:1, and 1:2 effector:target (E:T) ratios, respectively) in 96-well plates at 37° C. in the presence of 0.05 μM caspase-3/7 red (Incucyte, Essence BioScience). Plates ran on the Incucyte for 24-72 hours and measured for apoptosis activity via caspase cleavage and comparison of relative confluency. Following the assay, supernatants were collected for ELISA. Total GFP integrated intensity (Total GCU×μm²/Image) was assessed as a quantitative measure of live, GFP+ tumor cells. Values were normalized to the t=0 measurement.

Cytokine Secretion Assays. Cell supernatant collected from cell cytotoxicity assays was thawed and plated in triplicate for each condition. IFN-γ and IL-2 levels were determined using ELISA kits according to the manufacturer's protocol (BioLegend).

Expression, refolding, and purification of recombinant peptide/HLA molecules. HLA-A*02:01 and HLA-A*24:02 constructs for bacterial expression were cloned into pET24a+ plasmid. DNA plasmids encoding HLA-A*02:01 (heavy chain), HLA-A*24:02 (heavy chain), and human β2M (light chain) were transformed into E. coli BL21-DE3 (Novagen), expressed as inclusion bodies and refolded using previously described methods (Garboczi et al., 1992). E. coli cells were grown in autoinduction media for (16-18 hours) (Studier, 2014). Afterward, the E. coli cells were harvested by centrifugation and resuspended with 25 mL BugBuster (Milipore Sigma) per liters of culture. The cell lysate was sonicated and subsequently pelleted by centrifugation (5,180×g for 20 minutes at 4° C.) to collect inclusion bodies. The inclusion bodies were washed with 25 mL of wash buffer (100 mM Tris pH 8.0, 2 mM EDTA, and 0.01% v/v deoxycholate), sonicated, and pelleted by centrifugation. A second wash was done using 25 mL of Tris-EDTA buffer (100 mM Tris pH 8.0 and 2 mM EDTA). The solution was once again resuspended by sonication then centrifuged. The inclusion bodies were then solubilized by resuspension with 6 mL of resuspension buffer (100 mM Tris pH 8.0, 2 mM EDTA, 0.1 mM DTT, and 6 M guanidine-HCl). Solubilized inclusion bodies of the heavy and light chain were mixed in a 1:3 molar ratio and then added dropwise over 2 days to 1 L of refolding buffer (100 mM Tris pH 8.0, 2 mM EDTA, 0.4 M arginine-HCl, 4.9 mM L-glutathione reduced, and 0.57 mM L-glutathione oxidized) containing 10 mg of synthetic peptide at >98% purity confirmed by mass-spec (Genscript). Refolding was allowed to proceed for 4 days at 4° C. without stirring. Following this incubation period, the refolding mixture was dialyzed into the size-exclusion buffer (25 mM Tris pH 8.0 and 150 mM NaCl). After dialysis, the sample was concentrated first using a Labscale Tangential Flow Filtration system and then using an Amicon Ultra-15 Centrifugal 10 kDa MWCO Filter Unit (Millipore Sigma), to a final volume of 5 mL. Purification was performed using size-exclusion chromatography on a HiLoad 16/600 Superdex 75 column. After size-exclusion, the sample was further purified by anion exchange chromatography using a MonoQ 5/50 GL column and a 0-100% gradient of buffer A (25 mM Tris pH 8.0 and 50 mM NaCl) and buffer B (25 mM Tris pH 8.0 and 1 M NaCl). The purified protein was exhaustively exchanged into 20 mM sodium phosphate pH 7.2 and 50 mM NaCl. The final sample was validated using SDS-PAGE to confirm the formation of a pMHC complex containing both the heavy and light chains.

Immunohistochemistry. CD3 (Dako A0452), PHOX2B (Abcam ab183741), and HLA-ABC (Abcam ab70328) antibodies were used to stain formalin fixed paraffin embedded tissue slides. Staining was performed on a Bond Max automated staining system (Leica Biosystems). The Bond Refine polymer staining kit (Leica Biosystems, DS9800) was used. The standard protocol was followed with the exception of the primary antibody incubation which was extended to 1 hour at room temperature. CD3, PHOX2B, and HLA-ABC antibodies were at 1:100, 1:500, and 1:1200 dilutions respectively. Antigen retrieval was performed with E1 (Leica Biosystems) retrieval solution for 20 min (E2 for PHOX2B). Slides were rinsed, dehydrated through a series of ascending concentrations of ethanol and xylene, then coverslipped. Stained slides were then digitally scanned at 20× magnification on an Aperio CS-O slide scanner (Leica Biosystems).

Murine PC-CAR T cell preclinical trials. NOD SCID Gamma (NSG) female (6-8 weeks of age) mice from Jackson Laboratories (stock number 005557) were used to propagate subcutaneous xenografts. All mice were maintained under barrier conditions and experiments were conducted using protocols and conditions the IACUC at the Children's Hospital of Philadelphia. Treatment was initiated via lateral tail intravenous injection. Dose administered was 100 ul per animal of vehicle or CAR T cells as a single treatment. Treatment was administered when tumor volumes reached 150 mm³-250 mm³. Tumor volume and survival were monitored bi-weekly measurements until the tumors reached a size of 2.0 cm³ or mice showed signs of graft versus host disease. Animals were removed from study and studies terminated following onset of GVHD when animals display hunched posture, rapid breathing, urine staining, weight loss and a body condition score of 2, as determined by visual inspection. Onset of GVHD is defined as urine staining and weight loss of 20% or weight loss of 10-15% if accompanied by hunched posture, labored breathing, or poor body condition.

### Example 4—Results

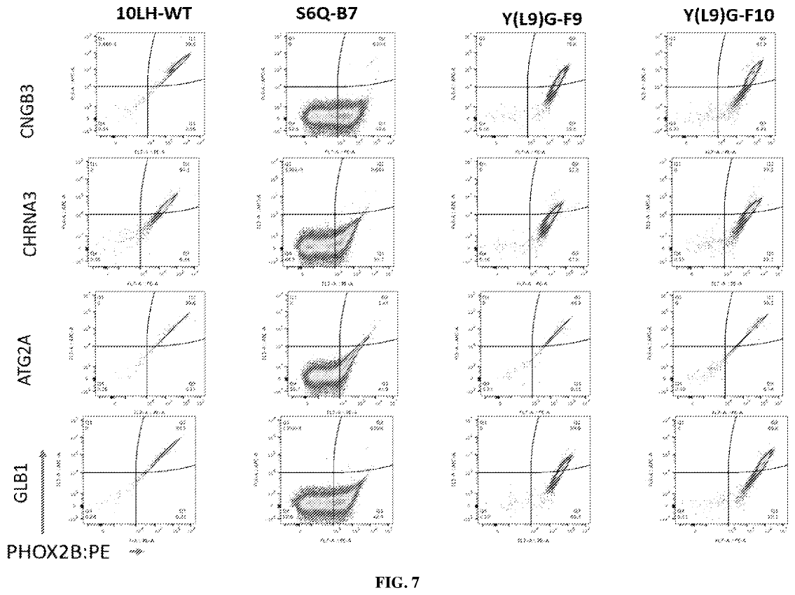

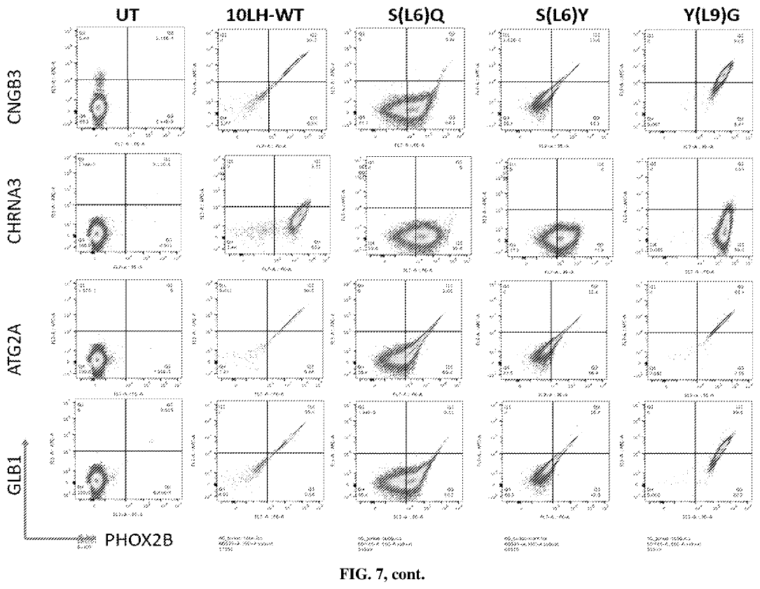

The inventors have previously developed a new class of chimeric antigen receptors (CAR) applied to a peptide derived from the PHOX2B protein in neuroblastoma, a childhood cancer. Subsequently, the inventors performed two orthogonal experiments that suggest improvements to the CAR that should improve efficacy/potency as well as safety. The first experiment was solving the crystal structure of the PHOX2B pMHC-scFv complex, and performing a series of surface plasmon resonance (SPR) binding experiments using variants of the original PHOX2B/HLA-A*24:02 target encompassing different peptides, HLA allotypes, or point mutations of HLA-A*24:02, to investigate the molecular determinants of cross-reactivity with 10LH. An independent experiment was a saturation mutagenesis screen of the scFv CDR loops. This approach aims to directly alter the CDR/peptide contacts, in favor of PHOX2B and against the other cross-reactive peptides. This was shown by the S6Q and Y9G mutants on CDRL3 in the mutagenesis screen, which aligns with the structural data as they hover right above the exposed peptide residues P6-P8. Indeed, the tetramer staining data of FIG. 7 show that S6Q and Y9G do not abrogate cross-reactivity with 3 out of 4 off-target peptides, and reduce overall binding to the target PHOX2B complex as evidence by an order of magnitude decrease in staining.

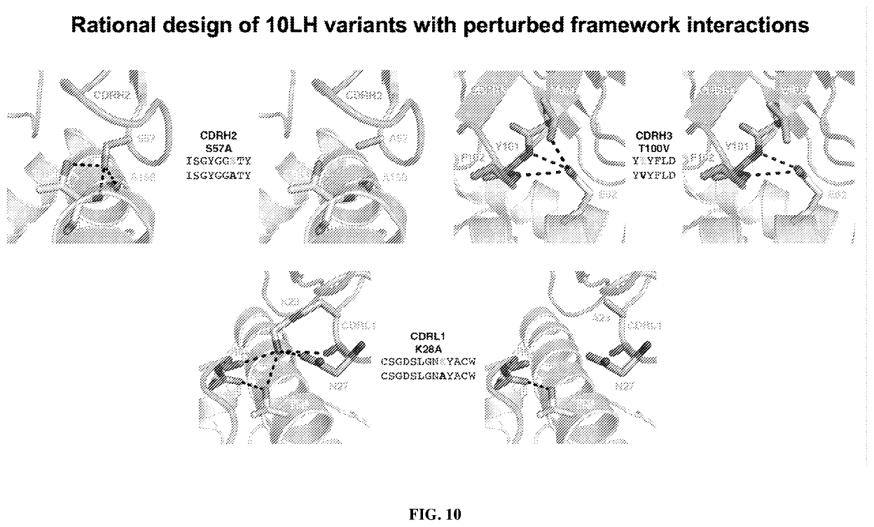

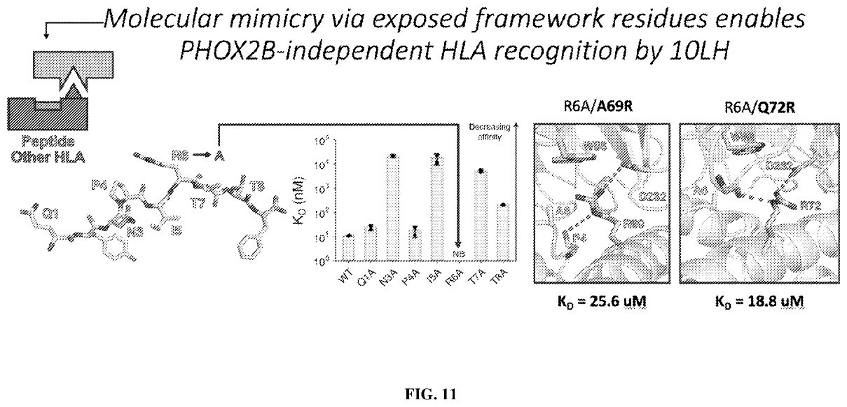

An alternative approach to minimize off-target cross reactivity by perturbing key interactions with HLA “framework” residues revealed by the crystal structure of the complex (FIG. 10). This strategy is leveraging the finding from the inventors' SPR binding studies that cross-reactive interactions with peptides (e.g., ATG2A) or HLAs with off-target peptides (e.g., HLA-C*07:02) are suboptimal (micromolar range) binders (vs nanomolar range interaction with HLA-A*24:02 PHOX2B). This is further consistent with structural modeling of the 10LH complexes with HLA-A*24:02^{A69R}/PHOX2B^{R6A} (approx. 26 micromolar K_{D} as measured by SPR) where cross-reactivity arises from imperfect molecular mimicry of the parental (HLA-A*24:02/PHOX2B) interface (FIG. 11). Since the 69R polymorphism can be found at all HLA-C* alleles, this mimicry mechanism could lead to off-target cross-reactivities whereby 10LH recognizes HLA-C* complexes presenting peptides not related to PHOX2B. Here, the inventors aimed to introduce point mutations on 10LH which weaken the HLA:10LH interface based on the 3D complex structure, such that any further perturbations to the interaction surface would abrogate off-target binding. This would likely also weaken interactions with the target HLA-A*24:02/PHOX2B target, from nanomolar to micromolar range K_{D} (dissociation constant); however, the inventors' SPR binding data and killing assay results together suggest that up to approx. 20 micromolar K_{D} interactions with pHLAs can elicit potent CAR-T killing (e.g., as seen for A*23:01/PHOX2B), dependent upon antigen density on the surface of the presenting cell. Based on this strategy, three mutation sites were selected based on the structure for further evaluation (FIG. 10).

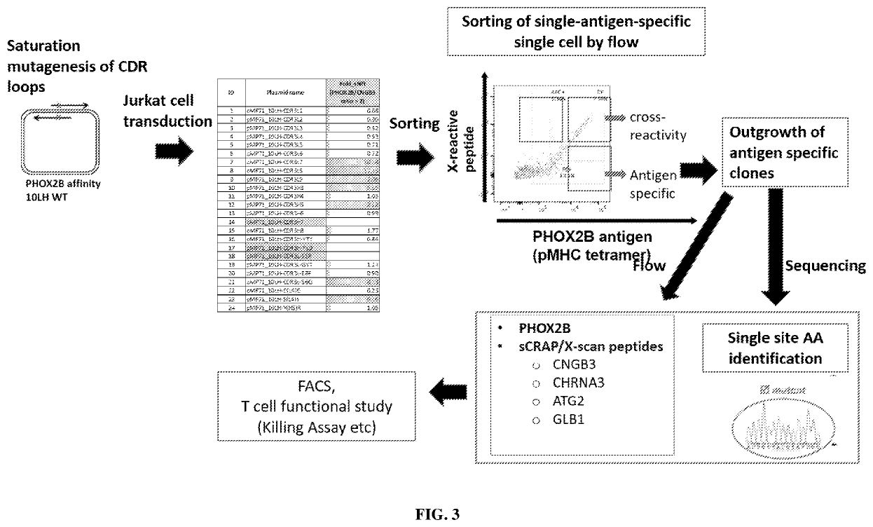

Workflow of site directed mutagenesis screening (FIG. 3). PCR amplification is used to mutate key residues of the CDR loops of the CAR scFv to all amino acids. After FACS sorting of single antigen-specific clones, clones are sequenced for mutation identification and functional validation of cytotoxicity and specificity.

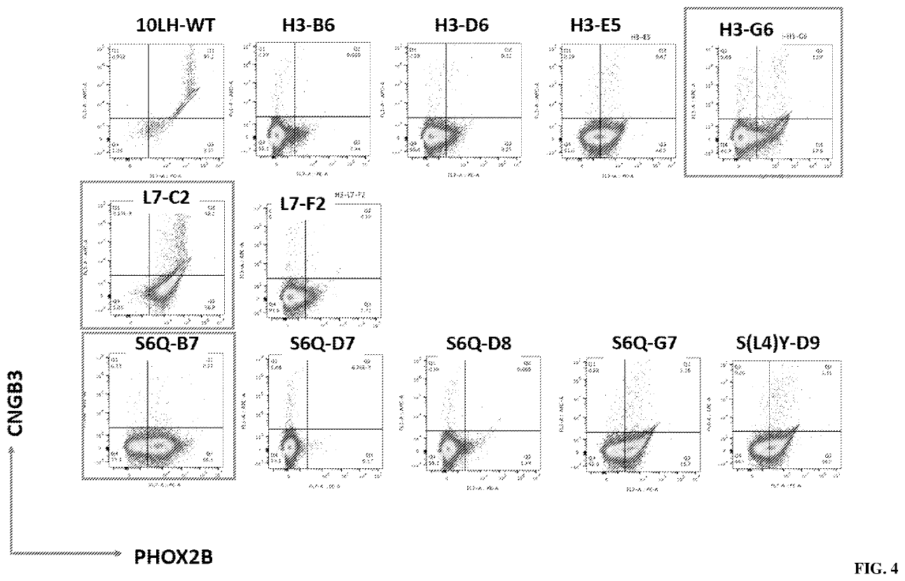

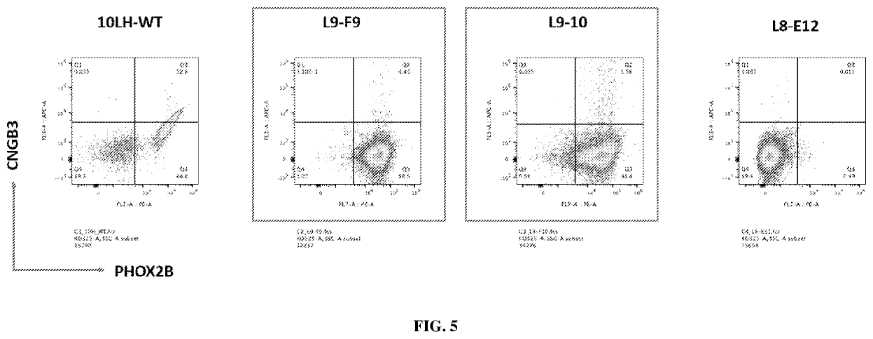

A screen of CDR3 loops identifies clones that resulted in shift toward single (FIG. 4). After mutagenesis of the CDR loops, single clones of Jurkat T cells expressing the 10LH.BBz chimeric antigen receptor (CAR) were stained with PHOX2B/HLA-A*24:02 and cross-reactive counterstain CNGB3/HLA-A*24:02 dextramers and analyzed via flow cytometry. Clones demonstrating PHOX2B antigen specificity were sorted using FACS. Clones mutated at position H3, L7, and S6Q demonstrated a shift towards PHOX2B antigen specificity compared to WT 10LH.BBz.

Two clones in Light chain CDR3 position 9 and 10 resulted in single-specificity binding to PHOX2B when compared to homologous CNGB3 peptide (FIG. 5). Following the procedures outlined previously, mutagenesis clones in light chain CDR3 position 9 and 10 demonstrated specificity to PHOX2B when counterstained with the WT cross-reactive CNGB3 peptide.

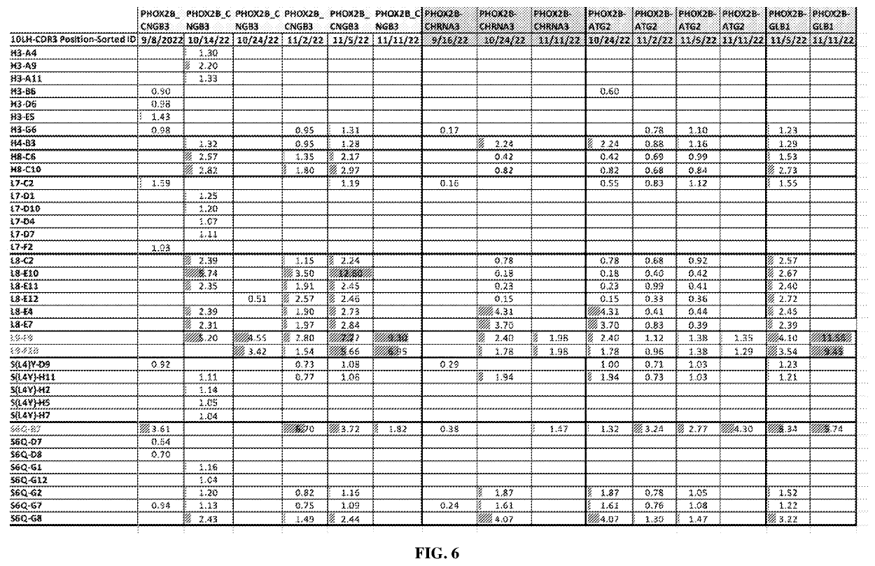

Quantifying the abrogation of cross-reactivity is shown in FIG. 6. Antigen specific population frequencies were compared between mutagenesis clones and 10LH.BBz WT CAR T cells using flow cytometry analysis. Fold shifts were calculated from PHOX2B antigen specific population frequencies divided by cross reactive, counterstain population frequencies.

Characterizing the shift in cross-reactivity of variants across sCRAP/X-scan peptides is shown in FIG. 7. Following the procedures outlined previously, mutagenesis clones were stained with PHOX2B and counterstained with known cross-reactive peptides determined from X-scan/ScanProsite and sCRAP.

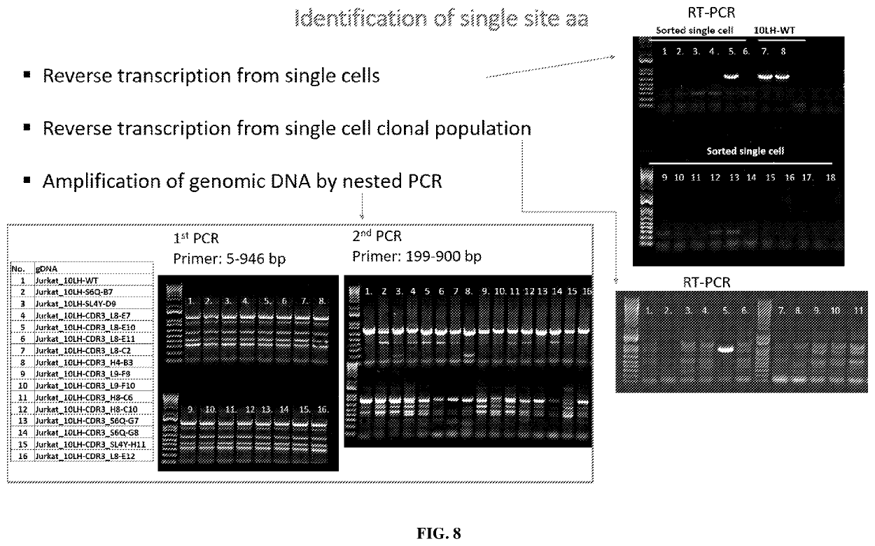

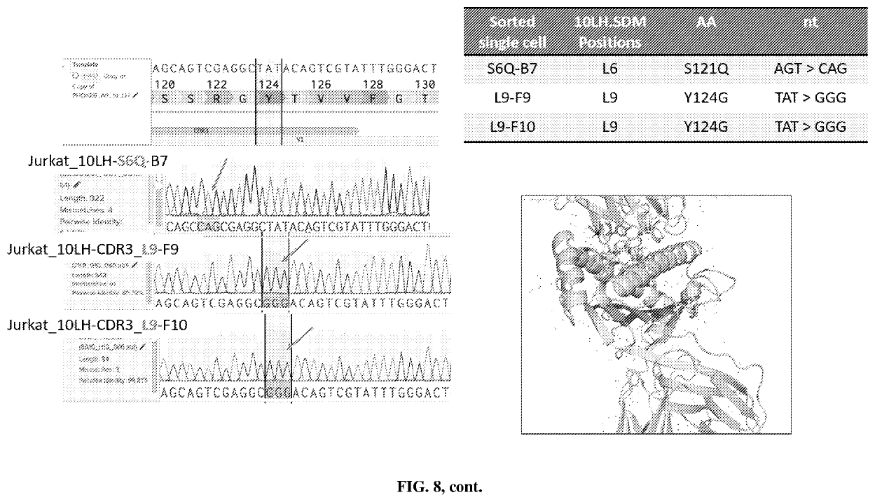

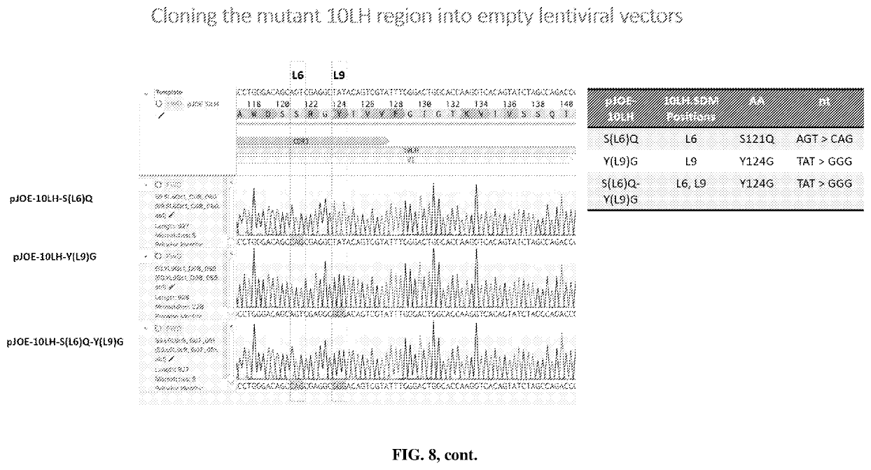

Mutagenesis amino acid identification was determined using PCR amplification methods. mRNA was recovered from single cells and populations grown from single cells, and reverse transcription was performed to synthesize cDNA. CDR loops of interest were then amplified using PCR. In addition, gDNA was recovered from single cells and populations grown from single cells, and nested PCR amplification was performed to amplify CDR loops of interest (FIG. 8). Mutants of interest demonstrating decreased cross-reactivity included point mutations S121Q (position L6) or Y124G (position L9).

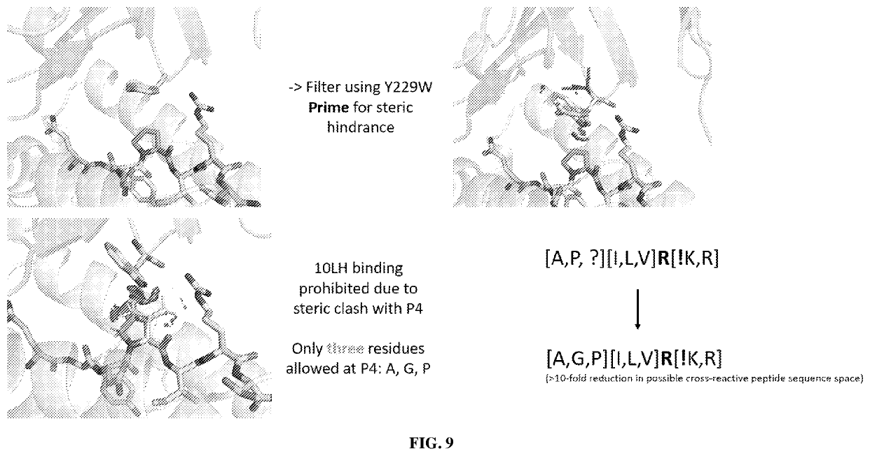

Building a P4 selectivity filter by point mutations of key 10LH CDR3H residues guided by the crystal structure of the 10LH/A*24:02/PHOX2B complex. Key 10LH residue interactions identified to be critical for binding to PHOX2B peptide residues by analysis of crystallographic contacts are shown in FIG. 9. Modeling of an exemplar Tyr to Trp amino acid substitution on the 10LH CDR using the crystal structure of the 10LH scFv in complex with PHOX2B/HLA-A*24:02. Structural modelling suggests that introducing a bulkier sidechain at this position would likely improve peptide specificity via steric hindrance. Specifically, upon the Y229W mutation, only three residues are allowed at position four of the PHOX2B 9mer QYNPIRTTF: A, G, and P.

TABLE 2
Sequences of exemplary antigen-binding proteins

| Name | SEQ ID NO: | Sequence |
|---|---|---|
| PHOX2B peptide | 1 | QYNPIRTTF |
| PHOX2B | 2 | MYKMEYSYLNSSAYESCMAGMDTSSLASAYADFSS |
| | | CSQASGFQYNPIRTTFGATSGCPSLTPGSCSLGTLRDH |
| | | QSSPYAAVPYKLFTDHGGLNEKRKQRRIRTTFTSAQL |
| | | KELERVFAETHYPDIYTREELALKIDLTEARVQVWFQ |
| | | NRRAKFRKQERAAAAAAAAAKNGSSGKKSDSSRDD |
| | | ESKEAKSTDPDSTGGPGPNPNPTPSCGANGGGGGGPS |
| | | PAGAPGAAGPGGPGGEPGKGGAAAAAAAAAAAAA |
| | | AAAAAAAGGLAAAGGPGQGWAPGPGPITSIPDSLGG |
| | | PFASVLSSLQRPNGAKAALVKSSMF |
| Clone 10 | 3 | SRNGGDGQSVLTQPPSVSVSPGQTASITCSGDSLGNK |
| (RU141-10) scFv | | YACWYQQKPGQSPVLVIYQDSKRPSGIPERFSGSNSG |
| | | NTATLTISGTQAMDEADYYCQAWDSSRGYTVVFGT |
| | | GTKVTVSSQTGGSGGGGSGGGGSGGGGSEVQLLESG |
| | | GGLVQPGGSLRLSCAASGFTFDSYAMSWVRQAPGK |
| | | GLEWVSAISGYGGSTYYADSVKGRFTISRDNSKNTL |
| | | YLQMNSLRAEDTAVYYCAKYTYFLDAFDIWGQGTM |
| | | VTVSSS |
| Clone 10 (RU141-10) | 4 | QSVLTQPPSVSVSPGQTASITCSGDSLGNKYACWYQ |
| scFv V_{L} | | QKPGQSPVLVIYQDSKRPSGIPERFSGSNSGNTATLTI |
| | | SGTQAMDEADYYCQAWDSSRGYTVVFGTGTKVTV |
| Clone 10 (RU141-10) | 5 | EVQLLESGGGLVQPGGSLRLSCAASGFTFDSYAMSW |
| scFv V_{H} | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSS |
| Clone 10LH (Clone | 6 | DSLGNKY |
| 10) V_{L} CDR1 | | |
| Clone 10LH (Clone | 7 | QDSKRPS |
| 10) V_{L} CDR2 | | |
| Clone 10LH (Clone | 8 | QAWDSSRGYTVV |
| 10) V_{L} CDR3 | | |
| Clone 10LH (Clone | 9 | GFTFDSYA |
| 10) V_{H} CDR1 | | |
| Clone 10LH (Clone | 10 | SGYGGS |
| 10) V_{H} CDR2 | | |
| Clone 10LH (Clone | 11 | YTYFLDAFD |
| 10) V_{H} CDR3 | | |
| PHOX2B 10LH PC- | 12 | MALPVTALLLPLALLLHAARPSRNGGDGQSVLTQPP |
| CAR 4-1bb/CD3z | | SVSVSPGQTASITCSGDSLGNKYACWYQQKPGQSPV |
| | | LVIYQDSKRPSGIPERFSGSNSGNTATLTISGTQAMDE |
| | | ADYYCQAWDSSRGYTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| CD8 leader | 13 | MALPVTALLLPLALLLHAARP |
| 10LH V_{L} | 14 | SRNGGDGQSVLTQPPSVSVSPGQTASITCSGDSLGNK |
| | | YACWYQQKPGQSPVLVIYQDSKRPSGIPERFSGSNSG |
| | | NTATLTISGTQAMDEADYYCQAWDSSRGYTVVFGT |
| | | GTKVTVSSQT |
| Linker | 15 | GGSGGGGSGGGGSGGGGS |
| 10LH V_{H} | 16 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSSSS |
| CD8 hinge | 17 | TTTPAPRPPTPAPTIASQPLSLRPEACRPAAGGAVHTR |
| | | GLDFACD |
| CD8 transmembrane | 18 | IYIWAPLAGTCGVLLLSLVITLYC |
| 4-1BB | 19 | KRGRKKLLYIFKQPFMRPVQTTQEEDGCSCRFPEEEE |
| | | GGCEL |
| CD3-zeta | 20 | RVKFSRSADAPAYKQGQNQLYNELNLGRREEYDVL |
| | | DKRRGRDPEMGGKPRRKNPQEGLYNELQKDKMALA |
| | | YSEIGMKGERRRGKGHDGLYQGLSTATKDTYDALH |
| | | MQALPPR |
| Clone 10-S121Q | 21 | SRNGGDGQSVLTQPPSVSVSPGQTASITCSGDSLGNK |
| scFv | | YACWYQQKPGQSPVLVIYQDSKRPSGIPERFSGSNSG |
| | | NTATLTISGTQAMDEADYYCQAWDSQRGYTVVFGT |
| | | GTKVTVSSQTGGSGGGGSGGGGSGGGGSEVQLLESG |
| | | GGLVQPGGSLRLSCAASGFTFDSYAMSWVRQAPGK |
| | | GLEWVSAISGYGGSTYYADSVKGRFTISRDNSKNTL |
| | | YLQMNSLRAEDTAVYYCAKYTYFLDAFDIWGQGTM |
| | | VTVSSS |
| Clone 10-S121Q | 22 | QSVLTQPPSVSVSPGQTASITCSGDSLGNKYACWYQ |
| scFv V_{L} | | QKPGQSPVLVIYQDSKRPSGIPERFSGSNSGNTATLTI |
| | | SGTQAMDEADYYCQAWDSQRGYTVVFGTGTKVTV |
| Clone 10-S121Q | 23 | EVQLLESGGGLVQPGGSLRLSCAASGFTFDSYAMSW |
| scFv V_{H} | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSS |
| Clone 10-S121Q V_{L} | 24 | DSLGNKY |
| CDR1 | | |
| Clone 10-S121Q V_{L} | 25 | QDSKRPS |
| CDR2 | | |
| Clone 10-S121Q V_{L} | 26 | QAWDSQRGYTVV |
| CDR3 | | |
| Clone 10-S121Q V_{H} | 27 | GFTFDSYA |
| CDR1 | | |
| Clone 10-S121Q V_{H} | 28 | SGYGGS |
| CDR2 | | |
| Clone 10-S121Q V_{H} | 29 | YTYFLDAFD |
| CDR3 | | |
| PHOX2B 10LH | 30 | MALPVTALLLPLALLLHAARPSRNGGDGQSVLTQPP |
| S121Q PC-CAR | | SVSVSPGQTASITCSGDSLGNKYACWYQQKPGQSPV |
| 4-1bb/CD3z | | LVIYQDSKRPSGIPERFSGSNSGNTATLTISGTQAMDE |
| | | ADYYCQAWDSQRGYTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-S121Q V_{L} | 31 | SRNGGDGQSVLTQPPSVSVSPGQTASITCSGDSLGNK |
| | | YACWYQQKPGQSPVLVIYQDSKRPSGIPERFSGSNSG |
| | | NTATLTISGTQAMDEADYYCQAWDSQRGYTVVFGT |
| | | GTKVTVSSQT |
| 10LH-S121Q V_{H} | 32 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSSSS |
| Clone 10-Y124G | 33 | SRNGGDGQSVLTQPPSVSVSPGQTASITCSGDSLGNK |
| scFv | | YACWYQQKPGQSPVLVIYQDSKRPSGIPERFSGSNSG |
| | | NTATLTISGTQAMDEADYYCQAWDSSRGGTVVFGT |
| | | GTKVTVSSQTGGSGGGGSGGGGSGGGGSEVQLLESG |
| | | GGLVQPGGSLRLSCAASGFTFDSYAMSWVRQAPGK |
| | | GLEWVSAISGYGGSTYYADSVKGRFTISRDNSKNTL |
| | | YLQMNSLRAEDTAVYYCAKYTYFLDAFDIWGQGTM |
| | | VTVSSS |
| Clone 10-Y124G | 34 | QSVLTQPPSVSVSPGQTASITCSGDSLGNKYACWYQ |
| scFv V_{L} | | QKPGQSPVLVIYQDSKRPSGIPERFSGSNSGNTATLTI |
| | | SGTQAMDEADYYCQAWDSSRGGTVVFGTGTKVTV |
| Clone 10-Y124G | 35 | EVQLLESGGGLVQPGGSLRLSCAASGFTFDSYAMSW |
| scFv V_{H} | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSS |
| Clone 10LH-Y124G | 36 | DSLGNKY |
| V_{L} CDR1 | | |
| Clone 10LH-Y124G | 37 | QDSKRPS |
| V_{L} CDR2 | | |
| Clone 10LH-Y124G | 38 | QAWDSSRGGTVV |
| V_{L} CDR3 | | |
| Clone 10LH-Y124G | 39 | GFTFDSYA |
| V_{H} CDR1 | | |
| Clone 10LH-Y124G | 40 | SGYGGS |
| V_{H} CDR2 | | |
| Clone 10LH-Y124G | 41 | YTYFLDAFD |
| V_{H} CDR3 | | |
| PHOX2B 10LH- | 42 | MALPVTALLLPLALLLHAARPSRNGGDGQSVLTQPP |
| Y124G PC-CAR | | SVSVSPGQTASITCSGDSLGNKYACWYQQKPGQSPV |
| 4-1bb/CD3z | | LVIYQDSKRPSGIPERFSGSNSGNTATLTISGTQAMDE |
| | | ADYYCQAWDSSRGGTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-Y124G V_{L} | 43 | SRNGGDGQSVLTQPPSVSVSPGQTASITCSGDSLGNK |
| | | YACWYQQKPGQSPVLVIYQDSKRPSGIPERFSGSNSG |
| | | NTATLTISGTQAMDEADYYCQAWDSSRGGTVVFGT |
| | | GTKVTVSSQT |
| 10LH-Y124G V_{H} | 44 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSSSS |
| Clone 10- | 45 | SRNGGDGQSVLTQPPSVSVSPGQTASITCSGDSLGNK |
| S121Q/Y124G scFv | | YACWYQQKPGQSPVLVIYQDSKRPSGIPERFSGSNSG |
| | | NTATLTISGTQAMDEADYYCQAWDSQRGYTVVFGT |
| | | GTKVTVSSQTGGSGGGGSGGGGSGGGGSEVQLLESG |
| | | GGLVQPGGSLRLSCAASGFTFDSYAMSWVRQAPGK |
| | | GLEWVSAISGYGGSTYYADSVKGRFTISRDNSKNTL |
| | | YLQMNSLRAEDTAVYYCAKYTYFLDAFDIWGQGTM |
| | | VTVSSS |
| Clone 10- | 46 | QSVLTQPPSVSVSPGQTASITCSGDSLGNKYACWYQ |
| S121Q/Y124G scFv | | QKPGQSPVLVIYQDSKRPSGIPERFSGSNSGNTATLTI |
| V_{L} | | SGTQAMDEADYYCQAWDSQRGGTVVFGTGTKVTV |
| Clone 10- | 47 | EVQLLESGGGLVQPGGSLRLSCAASGFTFDSYAMSW |
| S121Q/Y124G scFv | | VRQAPGKGLEWVSAISGYGGSTYYADSVKGRFTISR |
| V_{H} | | DNSKNTLYLQMNSLRAEDTAVYYCAKYTYFLDAFDI |
| | | WGQGTMVTVSS |
| Clone 10- | 48 | DSLGNKY |
| S121Q/Y124G V_{L} | | |
| CDR1 | | |
| Clone 10- | 49 | QDSKRPS |
| S121Q/Y124G V_{L} | | |
| CDR2 | | |
| Clone 10- | 50 | QAWDSQRGGTVV |
| S121Q/Y124G V_{L} | | |
| CDR3 | | |
| Clone 10- | 51 | GFTFDSYA |
| S121Q/Y124G V_{H} | | |
| CDR1 | | |
| Clonc 10- | 52 | SGYGGS |
| S121Q/Y124G V_{H} | | |
| CDR2 | | |
| Clone 10- | 53 | YTYFLDAFD |
| S121Q/Y124G V_{H} | | |
| CDR3 | | |
| PHOX2B 10LH | 54 | MALPVTALLLPLALLLHAARPSRNGGDGQSVLTQPP |
| S121Q/Y124G PC- | | SVSVSPGQTASITCSGDSLGNKYACWYQQKPGQSPV |
| CAR 4-1bb/CD3z | | LVIYQDSKRPSGIPERFSGSNSGNTATLTISGTQAMDE |
| | | ADYYCQAWDSQRGGTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-S121Q/Y124G | 55 | SRNGGDGQSVLTQPPSVSVSPGQTASITCSGDSLGNK |
| V_{L} | | YACWYQQKPGQSPVLVIYQDSKRPSGIPERFSGSNSG |
| | | NTATLTISGTQAMDEADYYCQAWDSQRGGTVVFGT |
| | | GTKVTVSSQT |
| 10LH-S121Q/Y124G | 56 | EVQLLESGGGLVQPGGSLRLSCAASGFTFDSYAMSW |
| V_{H} | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSSSS |
| Clone 10-T230V | 57 | QSVLTQPPSVSVSPGQTASITCSGDSLGNKYACWYQ |
| scFv | | QKPGQSPVLVIYQDSKRPSGIPERFSGSNSGNTATLTI |
| | | SGTQAMDEADYYCQAWDSSRGYTVVFGTGTKVTVS |
| | | SQTGGSGGGGSGGGGSGGGGSEVQLLESGGGLVQPG |
| | | GSLRLSCAASGFTFDSYAMSWVRQAPGKGLEWVSAI |
| | | SGYGGSTYYADSVKGRFTISRDNSKNTLYLQMNSLR |
| | | AEDTAVYYCAKYVYFLDAFDIWGQGTMVTVSS |
| Clone 10- T230V | 58 | QSVLTQPPSVSVSPGQTASITCSGDSLGNKYACWYQ |
| scFv V_{L} | | QKPGQSPVLVIYQDSKRPSGIPERFSGSNSGNTATLTI |
| | | SGTQAMDEADYYCQAWDSSRGYTVVFGTGTKVTV |
| Clone 10- T230V | 59 | EVQLLESGGGLVQPGGSLRLSCAASGFTFDSYAMSW |
| ScFv V_{H} | | VRQAPGKGLEWVSAISGYGGSTYYADSVKGRFTISR |
| | | DNSKNTLYLQMNSLRAEDTAVYYCAKYVYFLDAFD |
| | | IWGQGTMVTVSS |
| Clone 10-T230V V_{L} | 60 | DSLGNKY |
| CDR1 | | |
| Clone 10-T230V V_{L} | 61 | QDSKRPS |
| CDR2 | | |
| Clone 10-T230V V_{L} | 62 | CQAWDSSRGYTVV |
| CDR3 | | |
| Clone 10-T230V V_{H} | 63 | GFTFDSYA |
| CDR1 | | |
| Clone 10-T230V V_{H} | 64 | SGYGGS |
| CDR2 | | |
| Clone 10-T230V V_{H} | 65 | YVYFLDAFDI |
| CDR3 | | |
| PHOX2B 10LH- | 66 | MALPVTALLLPLALLLHAARPSRNGGDGQSVLTQPP |
| T230V PC-CAR | | SVSVSPGQTASITCSGDSLGNKYACWYQQKPGQSPV |
| 4-1bb/CD3z | | LVIYQDSKRPSGIPERFSGSNSGNTATLTISGTQAMDE |
| | | ADYYCQAWDSSRGYTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYVYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-T230V V_{L} | 67 | SRNGGDGQSVLTQPPSVSVSPGQTASITCSGDSLGNK |
| | | YACWYQQKPGQSPVLVIYQDSKRPSGIPERFSGSNSG |
| | | NTATLTISGTQAMDEADYYCQAWDSSRGYTVVIGT |
| | | GTKVTVSSQT |
| 10LH-T230V V_{H} | 68 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYVYFLDAFD |
| | | IWGQGTMVTVSSSS |
| Clone 10-K30A | 69 | QSVLTQPPSVSVSPGQTASITCSGDSLGNAYACWYQ |
| scFv | | QKPGQSPVLVIYQDSKRPSGIPERFSGSNSGNTATLTI |
| | | SGTQAMDEADYYCQAWDSSRGYTVVFGTGTKVTVS |
| | | SQTGGSGGGGSGGGGSGGGGSEVQLLESGGGLVQPG |
| | | GSLRLSCAASGFTFDSYAMSWVRQAPGKGLEWVSAI |
| | | SGYGGSTYYADSVKGRFTISRDNSKNTLYLQMNSLR |
| | | AEDTAVYYCAKYTYFLDAFDIWGQGTMVTVSS |
| Clone 10-K30A | 70 | QSVLTQPPSVSVSPGQTASITCSGDSLGNAYACWYQ |
| scFv V_{L} | | QKPGQSPVLVIYQDSKRPSGIPERFSGSNSGNTATLTI |
| | | SGTQAMDEADYYCQAWDSSRGYTVVFGTGTKVTV |
| Clone 10-K30A | 71 | EVQLLESGGGLVQPGGSLRLSCAASGFTFDSYAMSW |
| scFv V_{H} | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSS |
| Clone 10-K30A | 72 | DSLGNAY |
| V_{L} CDR1 | | |
| Clone 10-K30A | 73 | QDSKRPS |
| V_{L} CDR2 | | |
| Clone 10-K30A | 74 | QAWDSSRGYTVV |
| V_{L} CDR3 | | |
| Clonc 10-K30A | 75 | GFTFDSYA |
| V_{H} CDR1 | | |
| Clone 10-K30A | 76 | SGYGGS |
| V_{H} CDR2 | | |
| Clone 10-K30A | 77 | YTYFLDAFDI |
| V_{H} CDR3 | | |
| PHOX2B 10LH- | 78 | MALPVTALLLPLALLLHAARPSRNGGDGQSVLTQPP |
| K30A PC-CAR | | SVSVSPGQTASITCSGDSLGNAYACWYQQKPGQSPV |
| 4-1bb/CD3z | | LVIYQDSKRPSGIPERFSGSNSGNTATLTISGTQAMDE |
| | | ADYYCQAWDSSRGYTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGST |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-K30A V_{L} | 79 | SRNGGDGQSVLTQPPSVSVSPGQTASITCSGDSLGNA |
| | | YACWYQQKPGQSPVLVIYQDSKRPSGIPERFSGSNSG |
| | | NTATLTISGTQAMDEADYYCQAWDSSRGYTVVFGT |
| | | GTKVTVSSQT |
| 10LH-K30A V_{H} | 80 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYTYFLDAFDI |
| | | WGQGTMVTVSSSS |
| Clone 10-S187A | 81 | QSVLTQPPSVSVSPGQTASITCSGDSLGNKYACWYQ |
| scFv | | QKPGQSPVLVIYQDSKRPSGIPERFSGSNSGNTATLTI |
| | | SGTQAMDEADYYCQAWDSSRGYTVVFGTGTKVTVS |
| | | SQTGGSGGGGSGGGGSGGGGSEVQLLESGGGLVQPG |
| | | GSLRLSCAASGFTFDSYAMSWVRQAPGKGLEWVSAI |
| | | SGYGGATYYADSVKGRFTISRDNSKNTLYLQMNSLR |
| | | AEDTAVYYCAKYTYFLDAFDIWGQGTMVTVSS |
| Clone 10-S187A | 82 | QSVLTQPPSVSVSPGQTASITCSGDSLGNKYACWYQ |
| scFv V_{L} | | QKPGQSPVLVIYQDSKRPSGIPERFSGSNSGNTATLTI |
| | | SGTQAMDEADYYCQAWDSSRGYTVVFGTGTKVTV |
| Clone 10-S187A | 83 | EVQLLESGGGLVQPGGSLRLSCAASGFTFDSYAMSW |
| SCFv V_{H} | | VRQAPGKGLEWVSAISGYGGATYYADSVKGRFTISR |
| | | DNSKNTLYLQMNSLRAEDTAVYYCAKYTYFLDAFDI |
| | | WGQGTMVTVSS |
| Clone 10-S187A | 84 | DSLGNKY |
| V_{L} CDR1 | | |
| Clone 10-S187A | 85 | QDSKRPS |
| V_{L} CDR2 | | |
| Clone 10-S187A | 86 | QAWDSSRGYTVV |
| V_{L} CDR3 | | |
| Clone 10-S187A | 87 | GFTFDSYA |
| V_{H} CDR1 | | |
| Clone 10-S187A | 88 | SGYGGA |
| V_{H} CDR2 | | |
| Clone 10-S187A | 89 | YTYFLDAFDI |
| V_{H} CDR3 | | |
| PHOX2B 10LH- | 90 | MALPVTALLLPLALLLHAARPSRNGGDGQSVLTQPP |
| S187A PC-CAR 4- | | SVSVSPGQTASITCSGDSLGNKYACWYQQKPGQSPV |
| 1bb/CD3z | | LVIYQDSKRPSGIPERFSGSNSGNTATLTISGTQAMDE |
| | | ADYYCQAWDSSRGYTVVFGTGTKVTVSSQTGGSGG |
| | | GGSGGGGSGGGGSEVQLLESGGGLVQPGGSLRLSCA |
| | | ASGFTFDSYAMSWVRQAPGKGLEWVSAISGYGGAT |
| | | YYADSVKGRFTISRDNSKNTLYLQMNSLRAEDTAVY |
| | | YCAKYTYFLDAFDIWGQGTMVTVSSSSTTTPAPRPPT |
| | | PAPTIASQPLSLRPEACRPAAGGAVHTRGLDFACDIYI |
| | | WAPLAGTCGVLLLSLVITLYCKRGRKKLLYIFKQPFM |
| | | RPVQTTQEEDGCSCRFPEEEEGGCELRVKFSRSADAP |
| | | AYKQGQNQLYNELNLGRREEYDVLDKRRGRDPEMG |
| | | GKPRRKNPQEGLYNELQKDKMAEAYSEIGMKGERR |
| | | RGKGHDGLYQGLSTATKDTYDALHMQALPPR |
| 10LH-S187A V_{L} | 91 | SRNGGDGQSVLTQPPSVSVSPGQTASITCSGDSLGNK |
| | | YACWYQQKPGQSPVLVIYQDSKRPSGIPERFSGSNSG |
| | | NTATLTISGTQAMDEADYYCQAWDSSRGYTVVFGT |
| | | GTKVTVSSQT |
| 10LH-S187A V_{H} | 92 | EVQLLESGGGLVQPGGSLRLSCAASGFTFDSYAMSW |
| | | VRQAPGKGLEWVSAISGYGGATYYADSVKGRFTISR |
| | | DNSKNTLYLQMNSLRAEDTAVYYCAKYTYFLDAFDI |
| | | WGQGTMVTVSSSS |
| 10LH-Y229W V_{H} | 93 | EVQLLESGGGLVQPGGSLRLSCAASGFTFDSYAMSW |
| | | VRQAPGKGLEWVSAISGYGGSTYYADSVKGRFTISR |
| | | DNSKNTLYLQMNSLRAEDTAVYYCAKYT W FLDAF |
| | | DIWGQGTMVTVSS |

TABLE 3
Sequences for PHOX2B 10LH PC-CAR 4-1bb/CD3z

MALPVTALLLPLALLLHAARPSRNGGDGQSVLTQPPSVSVSPGQTASITCSGDSLGNKYACW
YQQKPGQSPVLVIYQDSKRPSGIPERFSGSNSGNTATLTISGTQAMDEADYYCQAWDSSRGY
TVVFGTGTKVTVSSQTGGSGGGGSGGGGSGGGGSEVQLLESGGGLVQPGGSLRLSCAASGF
TFDSYAMSWVRQAPGKGLEWVSAISGYGGSTYYADSVKGRFTISRDNSKNTLYLQMNSLRA
EDTAVYYCAKYTYFLDAFDIWGQGTMVTVSSSSTTTPAPRPPTPAPTIASQPLSLRPEACRPA
AGGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYD
ALHMQALPPR* (SEQ ID NO: 12)
Clone 10LH
CD8 leader: MALPVTALLLPLALLLHAARP (SEQ ID NO: 13)
10LH V_{L}
SRNGGDGQSVLTQPPSVSVSPGQTASITCSGDSLGNKYACWYQQKPGQSPVLVIYQDSKRPS
GIPERFSGSNSGNTATLTISGTQAMDEADYYCQAWDSSRGYTVVFGTGTKVTVSSQT (SEQ ID
NO: 14)
CDR1 DSLGNKY (SEQ ID NO: 6)
CDR2 QDSKRPS (SEQ ID NO: 7)
CDR3 QAWDSSRGYTVV (SEQ ID NO: 8)
Linker: GGSGGGGSGGGGSGGGGS (SEQ ID NO: 15)
10LH V_{H}
EVQLLESGGGLVQPGGSLRLSCAASGFTFDSYAMSWVRQAPGKGLEWVSAISGYGGSTYY
ADSVKGRFTISRDNSKNTLYLQMNSLRAEDTAVYYCAKYTYFLDAFDIWGQGTMVTVSSSS
(SEQ ID NO: 15)
CDR1 GFTFDSYA (SEQ ID NO: 9)
CDR2 SGYGGS (SEQ ID NO: 10)
CDR3 YTYFLDAFD (SEQ ID NO: 11)
CD8 hinge:
TTTPAPRPPTPAPTIASQPLSLRPEACRPAAGGAVHTRGLDFACD (SEQ ID NO: 17)
CD8 transmembrane:
IYIWAPLAGTCGVLLLSLVITLYC (SEQ ID NO: 18)
4-1BB:
KRGRKKLLYIFKQPFMRPVQTTQEEDGCSCRFPEEEEGGCEL (SEQ ID NO: 19)
CD3-zeta:
RVKFSRSADAPAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR* (SEQ ID
NO: 20)

TABLE 4
Sequences for PHOX2B 10LH-S121Q PC-CAR 4-1bb/CD3z

MALPVTALLLPLALLLHAARPSRNGGDGQSVLTQPPSVSVSPGQTASITCSGDSLGNKYACW
YQQKPGQSPVLVIYQDSKRPSGIPERFSGSNSGNTATLTISGTQAMDEADYYCQAWDSQRGY
TVVFGTGTKVTVSSQTGGSGGGGSGGGGSGGGGSEVQLLESGGGLVQPGGSLRLSCAASGF
TFDSYAMSWVRQAPGKGLEWVSAISGYGGSTYYADSVKGRFTISRDNSKNTLYLQMNSLRA
EDTAVYYCAKYTYFLDAFDIWGQGTMVTVSSSSTTTPAPRPPTPAPTIASQPLSLRPEACRPA
AGGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYD
ALHMQALPPR* (SEQ ID NO: 30)
Clone 10LH-S121Q
CD8 leader: MALPVTALLLPLALLLHAARP (SEQ ID NO: 13)
10LH-S121Q V_{L}
SRNGGDGQSVLTQPPSVSVSPGQTASITCSGDSLGNKYACWYQQKPGQSPVLVIYQDSKRPS
GIPERFSGSNSGNTATLTISGTQAMDEADYYCQAWDSQRGYTVVFGTGTKVTVSSQT (SEQ
ID NO: 31)
CDR1 DSLGNKY (SEQ ID NO: 24)
CDR2 QDSKRPS (SEQ ID NO: 25)
CDR3 QAWDSQRGYTVV (SEQ ID NO: 26)
Linker: GGSGGGGSGGGGSGGGGS (SEQ ID NO: 24)
10LH-S121Q V_{H}
EVQLLESGGGLVQPGGSLRLSCAASGFTFDSYAMSWVRQAPGKGLEWVSAISGYGGSTYY
ADSVKGRFTISRDNSKNTLYLQMNSLRAEDTAVYYCAKYTYFLDAFDIWGQGTMVTVSSSS
(SEQ ID NO: 32)
CDR1 GFTFDSYA (SEQ ID NO: 27)
CDR2 SGYGGS (SEQ ID NO: 28)
CDR3 YTYFLDAFD (SEQ ID NO: 29)
CD8 hinge:
TTTPAPRPPTPAPTIASQPLSLRPEACRPAAGGAVHTRGLDFACD (SEQ ID NO: 17)
CD8 transmembrane:
IYIWAPLAGTCGVLLLSLVITLYC (SEQ ID NO: 18)
4-1BB:
KRGRKKLLYIFKQPFMRPVQTTQEEDGCSCRFPEEEEGGCEL (SEQ ID NO: 19)
CD3-zeta:
RVKFSRSADAPAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR* (SEQ ID
NO: 20)

TABLE 5
Sequences for PHOX2B 10LH-Y124G PC-CAR 4-1bb/CD3z

MALPVTALLLPLALLLHAARPSRNGGDGQSVLTQPPSVSVSPGQTASITCSGDSLGNKYACW
YQQKPGQSPVLVIYQDSKRPSGIPERFSGSNSGNTATLTISGTQAMDEADYYCQAWDSSRGG
TVVFGTGTKVTVSSQTGGSGGGGSGGGGSGGGGSEVQLLESGGGLVQPGGSLRLSCAASGF
TFDSYAMSWVRQAPGKGLEWVSAISGYGGSTYYADSVKGRFTISRDNSKNTLYLQMNSLRA
EDTAVYYCAKYTYFLDAFDIWGQGTMVTVSSSSTTTPAPRPPTPAPTIASQPLSLRPEACRPA
AGGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYD
ALHMQALPPR* (SEQ ID NO: 42)
Clone 10LH
CD8 leader: MALPVTALLLPLALLLHAARP (SEQ ID NO: 13)
10LH V_{L}
SRNGGDGQSVLTQPPSVSVSPGQTASITCSGDSLGNKYACWYQQKPGQSPVLVIYQDSKRPS
GIPERFSGSNSGNTATLTISGTQAMDEADYYCQAWDSSRGGTVVFGTGTKVTVSSQT (SEQ ID
NO: 43)
CDR1 DSLGNKY (SEQ ID NO: 36)
CDR2 QDSKRPS (SEQ ID NO: 37)
CDR3 QAWDSSRGGTVV (SEQ ID NO: 38)
Linker: GGSGGGGSGGGGSGGGGS (SEQ ID NO: 15)
10LH V_{H}
EVQLLESGGGLVQPGGSLRLSCAASGFTFDSYAMSWVRQAPGKGLEWVSAISGYGGSTYY
ADSVKGRFTISRDNSKNTLYLQMNSLRAEDTAVYYCAKYTYFLDAFDIWGQGTMVTVSSSS
(SEQ ID NO: 44)
CDR1 GFTFDSYA (SEQ ID NO: 39)
CDR2 SGYGGS (SEQ ID NO: 40)
CDR3 YTYFLDAFD (SEQ ID NO: 41)
CD8 hinge:
TTTPAPRPPTPAPTIASQPLSLRPEACRPAAGGAVHTRGLDFACD (SEQ ID NO: 17)
CD8 transmembrane:
IYIWAPLAGTCGVLLLSLVITLYC (SEQ ID NO: 18)
4-1BB:
KRGRKKLLYIFKQPFMRPVQTTQEEDGCSCRFPEEEEGGCEL (SEQ ID NO: 19)
CD3-zeta:
RVKFSRSADAPAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR* (SEQ ID
NO: 20)

TABLE 6
Sequences for PHOX2B 10LH-S121Q/Y124G PC-CAR 4-1bb/CD3z

MALPVTALLLPLALLLHAARPSRNGGDGQSVLTQPPSVSVSPGQTASITCSGDSLGNKYACW
YQQKPGQSPVLVIYQDSKRPSGIPERFSGSNSGNTATLTISGTQAMDEADYYCQAWDSQRGG
TVVFGTGTKVTVSSQTGGSGGGGSGGGGSGGGGSEVQLLESGGGLVQPGGSLRLSCAASGF
TFDSYAMSWVRQAPGKGLEWVSAISGYGGSTYYADSVKGRFTISRDNSKNTLYLQMNSLRA
EDTAVYYCAKYTYFLDAFDIWGQGTMVTVSSSSTTTPAPRPPTPAPTIASQPLSLRPEACRPA
AGGAVHTRGLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYD
ALHMQALPPR* (SEQ ID NO: 54)
Clone 10LH-S121Q/Y124G
CD8 leader: MALPVTALLLPLALLLHAARP (SEQ ID NO: 13)
10LH-S121Q/Y124G V_{L}
SRNGGDGQSVLTQPPSVSVSPGQTASITCSGDSLGNKYACWYQQKPGQSPVLVIYQDSKRPS
GIPERFSGSNSGNTATLTISGTQAMDEADYYCQAWDSQRGGTVVFGTGTKVTVSSQT (SEQ
ID NO: 55)
CDR1 DSLGNKY (SEQ ID NO: 48)
CDR2 QDSKRPS (SEQ ID NO: 49)
CDR3 QAWDSQRGGTVV (SEQ ID NO: 50)
Linker: GGSGGGGSGGGGSGGGGS (SEQ ID NO: 15)
10LH-S121Q/Y124G V_{H}
EVQLLESGGGLVQPGGSLRLSCAASGFTFDSYAMSWVRQAPGKGLEWVSAISGYGGSTYY
ADSVKGRFTISRDNSKNTLYLQMNSLRAEDTAVYYCAKYTYFLDAFDIWGQGTMVTVSSSS
(SEQ ID NO: 56)
CDR1 GFTFDSYA (SEQ ID NO: 51)
CDR2 SGYGGS (SEQ ID NO: 52)
CDR3 YTYFLDAFD (SEQ ID NO: 53)
CD8 hinge:
TTTPAPRPPTPAPTIASQPLSLRPEACRPAAGGAVHTRGLDFACD (SEQ ID NO: 17)
CD8 transmembrane:
IYIWAPLAGTCGVLLLSLVITLYC (SEQ ID NO: 18)
4-1BB:
KRGRKKLLYIFKQPFMRPVQTTQEEDGCSCRFPEEEEGGCEL (SEQ ID NO: 19)
CD3-zeta:
RVKFSRSADAPAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR* (SEQ ID
NO: 20)

All of the compositions and methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### IX. REFERENCES

The following references and any other reference cited herein, to the extent that they provide exemplary procedural or other details supplementary to those set forth herein, are specifically incorporated herein by reference.
WO/2011/075761
WO/2013/023251
WO/2013/000023
Am J Hum Genet. 74: 761-764 (2004).
Andreatta et al., Bioinformatics 29, 8-14, (2012).
Alvarez et al., Mol Cell Proteomics 18, 2459-2477, (2019).
Barker et al., Nucleic Acids Res gkac1011 (2022).
Brady et al., Nature Communications 11, 5183, (2020).
Beasley et al., Biotechnol J 10, 783-789, (2015).
Burr et al., Cancer Cell 36, 385-401.e388 (2019).
Cheung et al., JAMA 307, 1062-1071, (2012).
Crittenden et al., Scientific Reports 8, 7012, (2018).
Danger et al., Development 130, 6635, (2003).
Dharia et al., Nature Genetics 53, 529-538, (2021).
Garboczi et al., Proc Natl Acad Sci USA (1992) 89:3429-33.
Germain & Margulies, Anna Rev Immunol 11, 403-450, (1993).
Hata et al., Archives of Pathology & Laboratory Medicine 139, 543-546, (2015).
Hung et al., Histopathology 71, 786-794 (2017).
Ilca et al., Cell Reports 29:1621-1632.e3 (2019).
Leko & Rosenberg, Cancer cell 38, 454-472, (2020).
Linette et al., Blood 122, 863-871, (2013).
Majzner & Mackall, C. L. Nature Medicine 25, 1341-1355, (2019).
Matthay et al., Nat Rev Dis Primers 2, 16078, (2016).
Maus et al., Molecular Therapy—Oncolytics 3, (2016).
Mirzaei et al., Front Immunol 8, 1850-1850 (2017).
Molenaar et al., Nature 483, 589-593, (2012).
Morgan et al., J Immunother 36, 133-151, (2013).
Mosse et al., Am J Hum Genet 75, 727-730, (2004).
Overall et al., Nature Communications 11, 1909, (2020).
Pattyn et al., Nature 399, 366-370, (1999).
Parkhurst et al., Molecular Therapy 19, 620-626 (2011).
Pei et al., Human Immunology 59:313-322 (1998).
Pei et al., Human Immunology 60:1293-1302 (1999).
Pugh et al., Nature genetics 45, 279-284, (2013).
Rosenthal et al., Nature 567, 479-485, (2019).
Schramm et al., Nature Genetics 47, 872-877, (2015).
Schumacher & Schreiber, Science 348, 69-74, (2015).
Sci Adv 7: eabd3311 (2021).
Shao et al., Nucleic Acids Res 46, D1237-D1247, (2018).
Shao et al., Bioinformatics for Cancer Immunotherapy: Methods and Protocols (ed Sebastian Boegel) 173-181 (Springer U S, 2020).
Studier, Methods Mol Biol 1091:17-32 (2014).
Tsherniak et al., Cell 170, 564-576.e516, (2017).
Woelfl et al., Cancer Immunol Immunother. Vol. 54 (2005).
Yossef et al., JCI Insight 3, (2018).
Yarmarkovich et al., Frontiers in Immunology 11, (2020).
Yarmarkovich et al., Nature 599:477-484 (2021).

1. A method of screening for cross-reactivity of a candidate peptide:HLA targeted therapeutic against off-target HLAs, the method comprising the steps of: (a) contacting an antigen-binding portion of the candidate peptide:HLA targeted therapeutic with a composition comprising human leukocyte antigens linked to a solid substrate, and (b) detecting binding of the antigen-binding portion of the candidate peptide:HLA targeted therapeutic to the solid substrate, wherein binding of the antigen-binding portion of the candidate peptide:HLA targeted therapeutic to the solid substrate is indicative of a cross-reactivity specific for said native human leukocyte antigens.

2. The method of claim 1, wherein the human leukocyte antigens linked to the solid substrate are peptide-loaded.

3. The method of claim 1, wherein the human leukocyte antigens linked to the solid substrate have been incubated with an excess of a target peptide prior art step (a).

4. The method of claim 3, wherein the target peptide is peptide to which the candidate peptide:HLA targeted therapeutic is targeted.

5. The method of claim 1, wherein the composition comprising the human leukocyte antigens linked to a solid substrate comprises at least 90% native human leukocyte antigens and at most 10% denatured human leukocyte antigens.

6. The method of claim 1, wherein the antigen-binding portion of the candidate peptide:HLA targeted therapeutic is an scFV.

7. The method of claim 1, wherein the human leukocyte antigens are selected from the group consisting of class I human leukocyte antigens, class II human leukocyte antigens and combinations thereof.

8. The method of claim 1, wherein the human leukocyte antigens are class I human leukocyte antigens.

9. The method of claim 1, wherein the solid substrate is selected from the group consisting of a plurality of beads, a plurality of microbeads, a plurality of microparticles, a plurality of microspheres, a well, a membrane, a polymer, a filter and a microarray and combinations thereof.

10. The method of claim 1, wherein the solid substrate is a plurality of microbeads.

11. The method of claim 1, wherein the solid substrate comprises a material selected from the group consisting of silica, gold, latex, polystyrene, polysulfone, hydrogel, polyvinyl chloride, glass, and combinations thereof.

12. The method of claim 1, wherein the solid substrate comprises a detectable label.

13. The method of claim 12, wherein the detectable label is a fluorescent dye, a radioactive label, a magnetic label, a bar code, or combinations thereof.

14. The method of claim 1, wherein said human leukocyte antigens are covalently linked to the solid substrate.

15. The method of claim 1, comprising a plurality of said solid substrates, wherein at least 90% of the human leukocyte antigens linked to a particular solid substrate of the plurality are of the same allele and each solid substrate of the plurality is linked to a different human leukocyte antigen allele with respect to the other solid substrates of the plurality.

16. The method of claim 15, wherein the native and denatured human leukocyte antigens are class I human leukocyte antigens.

17. The method of claim 15, wherein the plurality of solid substrates comprises four or more solid substrates, eight or more solid substrates, 16 or more solid substrates, or 32 or more solid substrates.

18-20. (canceled)

21. The method of claim 1, wherein the detecting binding of the antigen-binding portion of the candidate peptide:HLA targeted therapeutic is performed using flow cytometry.

22. The method of claim 1, wherein the detecting binding of the antigen-binding portion of the candidate peptide:HLA targeted therapeutic is performed using a secondary antibody.

23. The method of claim 22, wherein the secondary antibody comprises a label selected from the group consisting of a radioactive label, a fluorescent label, an enzymatic label, an avidin label, a biotin label and combinations thereof.

24. The method of claim 1, wherein the solid substrate comprises a plurality of microbeads or microparticles.

25. The method of claim 24, wherein the solid substrate comprises a plurality of microbeads, wherein each microbead of the plurality comprises a detectable label.

26. The method of claim 25, wherein the detectable label is a fluorescent dye, a radioactive label, a magnetic label, or a bar code.
